(19) United States
(12) Patent Application Publication
(10) Pub. No.: US 2009/0240520 A1
(43) Pub. Date: Sep. 24, 2009

(54) Health care system, biological information terminal

(76) Inventors: Kaori Takano, Chiba (JP); Ryuji Iijima, Chiba (JP); Kouichi Moirya, Chiba (JP); Shinichiro Miyahara, Chiba (JP); Norihiko Matsuo, Chiba (JP); Shigeru Uematsu, Chiba (JP); Masako Miyazaki, Edmonton (CA); Toshio Ohyanagi, Hokkaido (JP); James Miller, Edmonton (CA); Steven Sutphen, Edmonton (CA)

Correspondence Address: DINSMORE & SHOHL LLP, 1900 CHEMED CENTER, 255 EAST FIFTH STREET, CINCINNATI, OH, 45202, US

(21) Appl. No.: 10/562,043

(22) PCT Filed: Jun. 21, 2004
(86) PCT No.: PCT/JP04/09057

§ 371 Date: Jul. 9, 2007

(30) Foreign Application Priority Data
Jun. 23, 2003 (JP) 2003-178273

(51) Int. Cl. G06Q 50/00; G06F 15/16; G06Q 10/00; A61B 5/0205

(52) U.S. Cl. 705/2; 709/204; 705/9; 600/301

(57) ABSTRACT

A health care system capable of altering the schedule appropriately depending on the physical condition of a user, a biological information terminal, a schedule managing method, and a schedule managing program. Physical condition information is detected or an action is instructed based on first schedule information, and based on the detection results of physical condition information or the result of an action instruction, information of schedule to be executed is altered from the first information to second schedule information.

### FIELD OF THE INVENTION

The present invention is concerning to a health-care system to manage the health condition of a user, or users, and to those of a health-care device, living-body information terminal, the schedule management method, and the schedule management program to be used in the health-care system. And the present invention is concerning to the relay device and the managing device, both functioning as the health-care device.

### BACKGROUND OF THE INVENTION

The following systems have been suggested in the past to manage remotely schedules of such as the measurement temperature of patients or medication of a patient.

(1) There is a system in which a schedule of medication is registered in a management device beforehand and the management device notifies the user of medication at a fixed time according to the schedule by sounding alarms.

(2) There is a system in which a management device directs the terminal which is hold by a user, measurement of temperature, and medication schedule, and directs the terminal to notify the result of measurement, and the time of medication, and the system notifies a terminal of a doctor when a difference with those measurement results and scheduled values are not less than prefixed values.

(3) There is a system in which schedules of users are registered in a server, and the schedules are made checked and remade at more than one doctor's terminals. In this system, there is an input device by which a user inputs the execution state of the schedule.

However, by the management device in the above system (1), a doctor or a third party person is not able to know whether the patient actually took medicine as scheduled.

On the other hand, in the above system (2), since a user inputs medication time and the temperature measurement time, and the terminal notifies to the server as such, they can know whether the user behaves as prescribed in the schedule. But, the above system (2) notifies the terminal of a doctor only when there is a difference more than prefixed value between schedule and input information, by this system a health manager, such as doctor can only know whether prescription is observed as scheduled.

In the above system (2), notification is made to the terminal of a doctor, when the difference is more than the prefixed value, to request judgment by the doctor at an abnormal condition of a patient. In another word, at each time when an abnormal condition arises, it is necessary to send request to the terminal of the doctor at a remote place by connecting communication line.

On the other hand, in the above system (3), although more than one doctor can know the execution status of a user, when they try to make schedules by referring to execution status of schedules, when there are more than one object users for management, it will impose a sever burden to doctors and operators.

At an absence of doctors, when a necessity of change of schedule arises, there is a possibility that quick schedule change cannot be performed.

A health management system using communication lines for information exchange as in the above system (1), (2), and (3), generally speaking, at the time of abnormal communication line, or the patient is out of communication range, information exchanged is stopped. Thus, even if change of a certain prescription is needed to a patient, it cannot be coped with smoothly.

### SUMMARY OF THE INVENTION

The present invention was made in view of such a situation, and the purpose is to offer a health-care system which can change suitably the schedule responding to a user's condition, those used on the health-care system such as health-care device, a living-body information terminal, the schedule management method, and a schedule management program. Furthermore, it is to offer a relay device and a managing device functioning as a health-care device.

More specifically, the present invention is concerning the health-care device to communicate with the living-body information terminal that is to detect health condition information, which is information about the health condition of a user (for example, as described in the embodiment, blood pulse, acceleration, angular velocity, breathing, heartbeat, movement of body, snoring, blood sugar, and others), or is to direct said user to take actions about medical care (for example, as described in the embodiment, measurement of pulse, taking medicine, doing exercise, and inputting of information about the physical condition), and the health-care device characteristically comprises

a schedule storage means to store the first schedule information for said living body information terminal to detect health data or to issue directions to take actions and the second schedule information showing a schedule to be executed corresponding to the execution result,

a communication means to read said first schedule information from said schedule memory mean, and to send it to said living-body information terminal, to let it execute it,

a detection mean (as the action management part 25 in the embodiment) to detect from data transmitted from said living-body information terminal, the execution result of the schedule following said first schedule information, and

the schedule updating means (for example as the action management part 25 of the embodiment), to change a schedule information for said living-body information terminal to execute, from said first schedule information to the second schedule information stored in said storing means, according to the execution result detected by said detection means.

Here schedule information is compound of an action table information to detects health condition as information of user's health condition or to issue directions to take an action concerning medical treatment, and judgment table information in which action to process corresponding to the execution result of schedule is defined for each action expressed in the action table information.

Specifically, action table information includes a task number to identify each action, a number to identify a living-body information terminal which execute the action relating to the task number, a trigger condition for execution of the action or execution time of the action, content of what is really executed, duration of action time, the execution result corresponding to the action, and etc.

The judgment table information includes a name of the action, execution result of action possibly taken relating to the action name, content of physical movement corresponding to the execution result, and etc.

The action table information and the judgment table information can be treated as tables for example as in the FIG. 3 of the embodiment, and display of the content, creation, and modification are easily done.

The health-care device in the present invention is to communicate with the living-body information terminal which detects health condition information, which is information about the health condition of a user, or directs said user to take actions about medical care, characteristically comprises

a schedule storage means to store the first schedule information for said living-body information terminal to detect health data or to issue directions to take actions and the third schedule information showing a schedule corresponding to a direction inputted from exterior,

a communication means to read said first schedule information from said schedule memory means, and to send it to said living-body information terminal, to let it execute it,

a reception means to receive directions from exterior, and

the schedule updating means to change a schedule information to be transmitted to and executed by said living-body information terminal responding to directions received by said receiving part, from said first schedule information to said third schedule information.

Here, the health-care device of the present invention comprises detection means to detect execution result of the schedule following said third schedule information based on data transmitted from said living-body information terminal, and said schedule updating mean to change a schedule information for said living-body information terminal to execute it, according to the execution result detected by said detection means, from said third schedule information to the other schedule information.

The detection means in the health-care device of the present invention detects whether the action was executed following said schedule information, or whether the action was executed as well as the time of execution, or the measurement result of health information, or the measurement result of health information as well as the time of the measurement, or inputted information inputted from inputting means, or inputted information inputted from inputting means as well as the time of inputting said inputted information, or whether the detection of whether action was taken or not, is made within a prefixed continuation time, as the execution result of the schedule following said schedule information.

The schedule updating means in the health-care device of the present invention characteristically changes to schedule of said first schedule information when schedule of said second schedule information or said third schedule information is finished.

In addition, the schedule updating means characteristically transmits to said living-body information terminal said second schedule information or said third schedule information when schedule is changed from said first schedule to said second schedule or from said first schedule to said third schedule, or transmits said second schedule information or said third schedule information beforehand to said living-body information terminal, and at the change, the schedule is changed by sending an identification information assigned to said second schedule or said third schedule.

Said second schedule information or said third schedule information treated by the health-care device of the present invention is characteristically information derived by changing a part of said first schedule information.

Said first schedule information, said second schedule information, and said third schedule information processed in the health-care device of the present invention each has, the action table information to detect the health condition information concerning the health condition of a user, or to issue action directions to the user to take action regarding medical treatment, and the judgment table information to define actions or terminal's operations in order to act corresponding to the execution result of the schedule for each action indicated in the action table information, and said schedule updating means characteristically changes schedule information corresponding to the execution result of an action following said action table information based on information defined in said judgment table information.

The relay device in the present invention is a device materialized in the above health-care device.

Specifically, at least one living-body information terminal is connected to the relay device, and which is in turn connected to a managing device via the network in the health-care system, the relay device comprises,

a schedule storage means to store first schedule information which indicates schedule for said living-body information terminal to detect health conditions information or to issue action direction to take action, and the second schedule information which indicates schedule to be executed according to the execution result of schedule of the first schedule information,

a communication means to read schedule information from said schedule storage means and to transmit to said living-body information terminal to execute it,

a detection means to detect execution result of the schedule of said first schedule information by data transmitted from said living-body information terminal, and

a schedule updating means to change schedule information to be sent to and to be executed by said living-body information terminal from said first schedule information to said second schedule information according to the execution result detected by said detection means.

The relay device in the present invention is in healthcare system connected at least one living-body information terminal, and is connected to a managing device via the network, characteristically comprises,

a schedule storage means to store the first schedule information which indicates schedule for said living-body information terminal to detect health conditions or to issue action direction to take action, and the third schedule information which indicates schedule according to directions inputted from exterior,

a communication means to read schedule information from said schedule storage means and to transmit to said living-body information terminal to execute it,

a reception means to receive directions from exterior,

a schedule updating means to change schedule information to send to and to be executed by said living-body information terminal from said first schedule information to said third schedule information according to the directions received by said reception means.

And the managing device in the present invention is one of devices materialized in the above health-care device.

More specifically, the managing device is a device in healthcare system where the relay device is connected at least one living-body information terminal, and said relay device is connected to a managing device via the network, characteristically comprises,

a schedule storage means to store first schedule information which indicates schedule for said living-body information terminal to detect health conditions or to issue action direction to take action, and the second schedule information which indicates schedule to be executed according to the execution result of schedule of the first schedule information,

a communication means to read schedule information from said schedule storage means and to transmit to said living-body information terminal to execute it,

a detection means to detect execution result of the schedule of the first schedule information by data transmitted from said living-body information terminal through said relay device, and

a schedule updating means to change schedule information to send to and to be executed by said living-body information terminal from said first schedule information to said second schedule information according to the execution result detected by said detection means.

The managing device in the present invention is a device in healthcare system where the relay device is connected to at least one living-body information terminal, and said relay device is connected to a managing device via the network, characteristically comprises,

a schedule storage means to store first schedule information which indicates schedule for said living-body information terminal to detect health conditions or to issue action direction to take action, and the third schedule information which indicates schedule according to directions inputted from exterior,

a communication means to read schedule information from said schedule storage means and to transmit to said living-body information terminal to execute it,

a reception means to receive directions from exterior,

a schedule updating means to change schedule information to send to and to be executed by said living-body information terminal for execution from said first schedule information to said third schedule information according to the directions received by said reception means.

And the living-body information terminal in the present invention is a living-body information terminal which communicates with a health-care device, detects health information regarding the health condition of a user, and to said user, directs actions regarding health treatment, characteristically comprises,

a communication mean which receives first schedule information and second schedule information from said health-care device,

a schedule execution management means, to detect health condition or to issue action direction to take action, based on said first schedule information (for example as the schedule execution management part 15 of the embodiment),

a detection means to detect execution result of health condition detection or the directions of actions executed by said schedule execution management means (for example as the schedule execution management part 15 of the embodiment), and

a schedule change means (for example as the schedule execution management part 15 of the embodiment) to change the schedule information to be executed at said schedule execution management means based on the execution result detected by said detection means from said first schedule information to said second schedule information.

The living-body information terminal in the present invention, communicating with the health-care device, detects health condition concerning the health condition of a user, or issue directions to prompt said user to make action regarding medical treatment, characteristically comprises

a communication means to receive the first schedule information and the third schedule information from said health-care device,

a schedule execution management means to detect health conditions or to issue the directions to take action, following the first schedule information received by said communication means,

a reception means to receive directions from exterior,

a schedule updating means to change the schedule information to be executed at said schedule execution management means from said first schedule information to said third schedule information according to the directions received by said communication means.

Here, the living-body information terminal in the present invention comprises a schedule execution management means to detect health condition or issue action direction to take actions following said third schedule information and a detection means to detect the schedule execution result following said third schedule information, is characterized in that the schedule updating means changes schedule information to be executed from said third schedule information to other schedule information corresponding to the execution result of said detection means.

The detection means in the present invention in the living-body terminal, characteristically detects as the execution result of the schedule following said schedule information, whether action was executed following said schedule information, or whether said action was executed as well as the time of execution, or the measuring result of health condition, or measuring result of health condition as well as its measuring time, or inputted information inputted at the inputting means. or inputted information inputted by the inputted means as well as its inputting time, or whether the detection is made within the prefixed time, of detection of whether an action was taken.

The detection means in the living-body information terminal of the present invention characteristically detects measuring result of one of or both of pulse rate and blood sugar value, or the measurement time of the measurements following said schedule information as execution results of the schedule following said schedule information.

The schedule updating means in the living-body information terminal of the present invention characteristically changes schedule to said first schedule information when schedule of said second schedule information is completed, or schedule of said third schedule information is completed.

The schedule updating means of the present invention when schedule is changed form said first schedule information to said second schedule information or from said first schedule information to said third schedule information,

receives and changes said second schedule information or said third schedule information sent from said health-care device, or after letting the living-body information terminal have received the second schedule information or the third schedule information from said health-care device beforehand, and at the reception of identification information of the second schedule information or the third schedule information from said health-care device, changes schedule information to said second schedule information or said third schedule corresponding to the received identification information.

Said second schedule information or said third schedule information dealt in the living-body information terminal are information characteristically derived by changing a part of said first schedule information.

Said first schedule information, said second schedule information, and said third schedule information dealt in the living-body information terminal of the present invention, each comprises, an action table information to detect the health condition as information regarding user's health condition, and to show action directions to let the user perform action regarding medical treatment, and a judgment table information defining for each action in the action table, terminal's operations for treatments corresponding to execution results of the schedule, and is characterized in that said schedule updating means changes schedule information according to the result of execution following said action table information based on information defined in said judgment table information.

In addition, the living-body information terminal of the present invention characteristically possesses a judgment request means to request said health-care device to judge when the schedule execution result following said first schedule information, said second schedule information, and said third schedule information is judged abnormal using said judgment table information.

And the living-body information terminal of the present invention characteristically transmits and let other living-body information terminal display the schedule information or the execution result of the schedule information of its own when said multiple living-body information terminals are connected mutually communicatively.

Further, the health-care system of the present invention in which at least one living-body information terminal is connected to a relay device, and via said relay device connected to a managing device, when said living-body information terminal side is called lower side, and said management terminal side is called upper side, the upper side device characteristically stores information concerning all schedule in the lower side device as well as schedule information of lower device in cases lower side schedule information is changed by the upper side judgment.

And the health-care system of the present invention is connected to a health-care device and a living-body information terminal, which detects health condition information as the health condition of a user, or issues action directions for the user to take actions concerning medical treatment, and said health-care device characteristically comprises,

a schedule storage means to store the first schedule information for said living-body information terminal to detect health condition information or to show schedule to issue a direction to take action, and to store the second schedule information that specifies the schedule corresponding to the execution result of said first schedule,

a communication means to read said schedule information from said schedule storage means and to transmit to said living-body information terminal and let it execute the information,

a detection means to detect the schedule execution result following said schedule information based on data transmitted from said living-body information terminal, and

a schedule updating means to change the schedule information to transmit to and to be executed by said living-body information terminal from the first schedule information to the second schedule information based on the execution result detected by said detection means,

while said living-body information terminal comprises

a communication means to receive the first schedule information and the second schedule information from said health-care device,

a schedule execution managing device to detect health condition or to direct to take actions based on the first schedule information received by said communication means,

a detection means to detect the execution results of said schedule execution managing means to detect health condition or to direct to take actions, and

a schedule updating means to change the schedule information to be executed by said schedule execution managing means from said first schedule information to said second schedule information based on the execution result detected by said detection means.

And the health-care system in the present invention is connected to a health-care device and a living-body information terminal that detects health information concerning information of health condition of a user, or issues direction to let said user to take actions regarding medical treatment, and said health-care device characteristically comprises,

schedule storage means to store the first schedule information for said living-body information terminal to detect health condition information or to show schedule to issue a direction to take action, and to store the third schedule information to show the schedule corresponding to the direction inputted from exterior,

a communication means to read said first schedule information from said schedule storage means and to send to said living-body information terminal and let it execute it,

a reception part to receive directions from exterior, and

a schedule updating means to change schedule information to be transmitted to and to be executed by said receiving part from said first schedule information to said third schedule information corresponding to the direction received by said reception part,

while said living-body information terminal comprises,

a communication means to receive the first schedule information and the third schedule information from said health-care device,

a schedule execution management means to detect health condition or to issue directions to act based on schedule information received by said communication means,

a reception means to receive directions from exterior, and

a schedule updating means to change schedule information to be executed by said schedule management means from said first schedule information to said third schedule information corresponding to the direction received by said receiving mean.

In addition, the schedule management method in the health-care system in the present invention is a schedule execution method to detect health condition information as information concerning user's health condition, or to issue action directions for the user to take actions concerning medical treatment, is characterized,

to issue action directions to detect health condition information based on the first schedule information,

to detect the detection result of said health condition information, or the result of the action directions, and

to change schedule information to be executed corresponding to the detection result from said first schedule information to the second schedule information.

The schedule management program in the health-care system of the present invention characteristically is a computer program to execute an action according to the schedule management method in the above health-care system.

The schedule management method in the health-care system in the present invention is to detect health condition information as information concerning user's health condition, or to issue action directions for the user to take actions concerning medical treatment, is characterized,

to issue action directions to detect health condition information based on the first schedule information,

to receive direction from exterior, and

to change schedule information to be executed corresponding to the received direction from said first schedule information to the second schedule information.

The schedule management program in the health-care system of the present invention characteristically is a computer program to execute an action according to the schedule management method in the above health-care system.

In addition, the schedule management method used in the rely device in the health-care system is characterized,

to send to and to be executed by said living-body information terminal the first schedule information as a schedule for detection of health condition and issuing directions for actions,

to detect the execution result of action following the first schedule information sent from said living-body information terminal, and

to change the schedule information to be executed, corresponding to the execution result, from said first schedule information to the second schedule information.

The schedule management program used in the relay device in the health-care system of the present invention characteristically is a computer program to execute an action according to the schedule management method used in a relay device in the above health-care system.

The schedule management method used in a rely device in the health-care system is characterized,

to send to and to be executed by said living-body information terminal the first schedule information as a schedule for detection of health condition and issuing directions for actions,

to receive directions from exterior, and

to change the schedule information to be executed by said living-body information terminal, corresponding to said detection result, from said first schedule information to the third schedule information.

The schedule management program used in a relay device in the health-care system of the present invention characteristically is a computer program to execute an action according to the schedule management method used in a relay device in the above health-care system.

In addition, the schedule management method used in a managing device in the health-care system is characterized

to send to and to be executed by said living-body information terminal the first schedule information as a schedule for detection of health condition and issuing directions for actions,

to detect execution result of the action following said first schedule information, based on data transmitted from said living-body information terminal via said relay device, and

to change schedule information to be executed by said living-body information terminal via said relay, corresponding to the execution result, from said first schedule information to the second schedule information.

The schedule management program used in a managing device in the health-care system of the present invention characteristically is a computer program to execute an action according to the schedule management method used in a managing device in the above health-care system.

The schedule management method used in a managing device in the health-care system is characterized

to send to and to be executed by said living-body information terminal the first schedule information as a schedule for detection of health condition and issuing directions for actions,

to receive directions from exterior, and

to change schedule information to be executed by said living-body information terminal based on the received directions, from said first schedule information to the third schedule information.

The schedule management program used in a managing device in the health-care system of the present invention characteristically is a computer program to execute an action according to the schedule management method used in a managing device in the above health-care system.

In addition, the schedule management method used in the living-body information terminal in the health-care system is characterized to detect health condition or to issue directions for actions, based on the first schedule information,

to detect the detection result of said health condition, or result of the issuing directions for actions,

to change schedule information to be executed corresponding to the detection result, from said first schedule information to the second schedule information.

The schedule management program used in a living-body information terminal in the health-care system of the present invention characteristically is a computer program to execute an action according to the schedule management method used in a living-body information terminal in the above health-care system.

The schedule management method used in the living-body information terminal in the health-care system is characterized

to detect health condition or to issue directions for actions, based on the first schedule information,

to receive directions from exterior, and

to change schedule information to be executed corresponding to the received directions, from said first schedule information to the third schedule information.

The schedule management program used in a living-body information terminal in the health-care system of the present invention characteristically is a computer program to execute an action according to the schedule management method used in a living-body information terminal in the above health-care system.

### BRIEF DESCRIPTION OF THE DRAWINGS

FIG. 1 is an outline figure depicting the health-care system configuration as the first embodiment of the invention.

FIG. 2 is an outline block diagram for explaining the configuration of S Terminal 1.

FIG. 3 is a drawing for explaining action table information and judgment table information.

FIG. 4 is an outline block diagram showing configuration of C Server 2.

FIG. 5 is a drawing for explaining the action table information and the judgment table information stored in the memory part 24.

FIG. 6 is a flow chart for explaining the outline of functions of S Terminal 1 and C Server 2.

FIG. 7 is a flow chart for explaining the function of C Server 2.

FIG. 8 is a flow chart for explaining the function of C Server 2.

FIG. 9 is a flow chart for explaining the function of C Server 2.

FIG. 10 is a flow chart for explaining the function of C Server 2.

FIG. 11 is a flow chart for explaining the function of C Server 2.

FIG. 12 is a flow chart for explaining the function of C Server 2.

FIG. 13 is a flow chart for explaining the function of C Server 2.

FIG. 14 is a flow chart for explaining the function of S Terminal 1.

FIG. 15 is a flow chart for explaining the function of S Terminal 1.

FIG. 16 is a flow chart for explaining the function of S Terminal 1.

FIG. 17 is a flow chart for explaining the function of S Terminal 1.

FIG. 18 is a flow chart for explaining the function of S Terminal 1.

FIG. 19 is a flow chart for explaining the function of S Terminal 1.

FIG. 20 is an outline figure depicting the health-care system configuration as the second embodiment of the present invention.

FIG. 21 is an outline block diagram for explaining configuration of P Server 4.

FIG. 22 is a drawing for explaining the state of storing action table information and judgment table information.

FIG. 23 is a drawing for explaining distribution of functions between C Server 2 and P Server 4.

FIG. 24 is a drawing showing the action table information and the judgment table information which are stored by the living-body information terminal.

FIG. 25 is a drawing showing the action table information and the judgment table information which are stored in P Server 4.

FIG. 26 is a drawing showing the action table information stored in C Server 2.

FIG. 27 is a drawing showing the judgment table information stored in C Server 2.

FIG. 28 is a drawing for explaining the composition of the health-care system in other embodiment of the present invention.

FIG. 29 is an outline block diagram showing the composition of the health-care system in the third embodiment of the present invention.

FIG. 30 is a drawing for explaining one example of the action table information of other embodiment.

FIG. 31 is a drawing showing one example of the judgment table information of the other embodiment.

FIG. 32 is a flow chart for explaining the operation of the health-care system of the other embodiment.

FIG. 33 is a drawing showing one example of the action table information of the other embodiment.

FIG. 34 is a drawing showing one example of the action table information of the other embodiment.

FIG. 35 is a drawing showing one example of the schedule information for explaining the other embodiment for the generation of the action table information after change.

FIG. 36 is a drawing for explaining S Terminal 1 of the fourth embodiment of the present invention.

FIG. 37 is a graph referred for explaining the sixth embodiment of the present invention.

### DESCRIPTION OF THE PREFERRED EMBODIMENTS

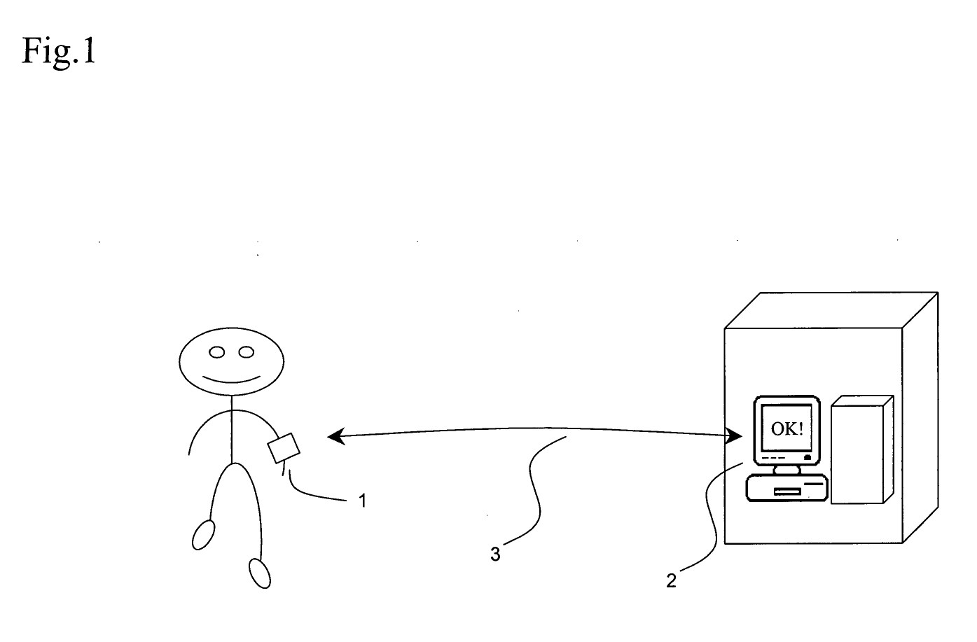

In below, the health-care system which applied to the health-care device and the living-body information terminal, in one embodiment of this invention is explained with reference to drawings. FIG. 1 is the outline diagram showing the composition of the health-care system. In this figure, the health-care system is that a living-body information terminal 1 (hereafter called “S terminal”), and a central server as an example of the health-care device 2 (hereafter called “C server”) is connected by a communication circuit 3. This communication circuit 3 is, for example, a public line network, a dedicated line, LAN (Local Area Network), or one or more such sets combined, and communication is performed by a cable or radio. Moreover, by using an-all-time connected or broadband communications, between C server 2 and S Terminal 1, it is possible to make the use of S terminal 1 less dependent on the location of the user.

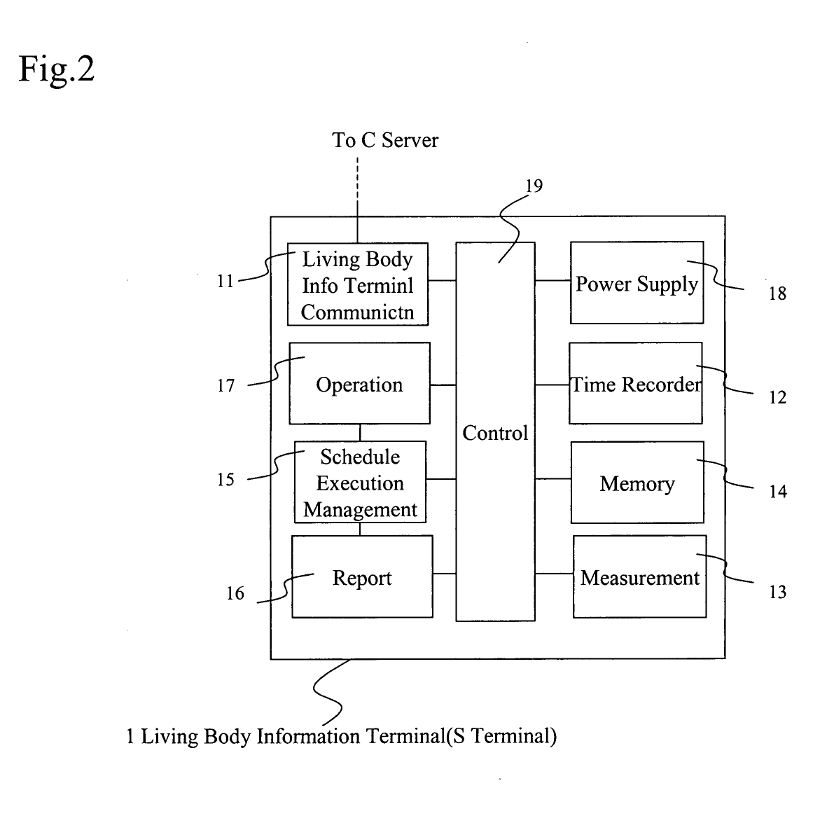

Next S Terminal 1 is explained. The FIG. 2 is a block diagram explaining the configuration of S Terminal 1. In this embodiment, the S Terminal 1 is able to measure the user's pulse, and it is a wrist-watch type terminal. In this figure, the living-body information terminal communication part 11 (denoted by “Living Body Info Terminal Communictn”), communicates with the central server 2 through the communication circuit 3. The time recorder 12 (noted as “Time Recorder”), based from the out-put of the internal clock circuit, controls time. Measurement part 13 (noted as “Measurement”) measures the user's pulse. Memory part 14 (noted as “Memory”) stores action table information, and judgment table information transmitted from C server 2, by receiving information at the living body information terminal communication part 11. About this action table information and judgment table information, will be explained later.

The schedule execution management part 15 (noted as “Schedule Execution Management”) controls schedule based on the action table information, referring to time output from the time record part 12. Report part 16 (noted as “Report”) is comprising a speaker and display device, for example, such as liquid-crystal display device, etc. and based on the instruction of control part 19, gives off alarm sound, and displays a message, etc. Operation part 17 (noted as “Operation”) is an input apparatus, such as, a touch panel, and a ten key. Power supply 18 is, a battery, or a battery that can be charged, and supplies power to parts in S Terminal 1. Control part 19 (noted as “Control”) controls transmissions of data between parts, and the voltage of the power supply to parts in S Terminal.

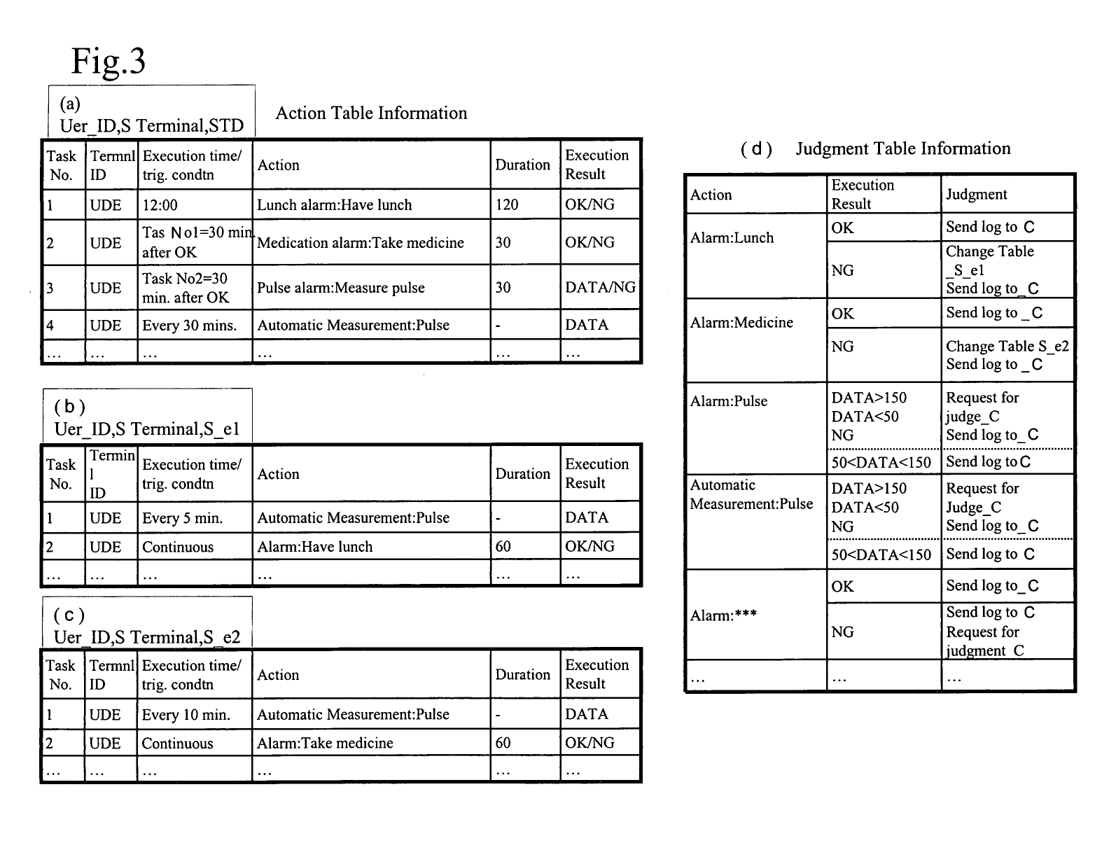

FIG. 3 is to explain the action table information, and the judgment table information. In the figure, the action table information stores task numbers to identify an action (in FIG. 3, corresponding to “Task No”), an terminal ID to identify S Terminal 1, an trigger condition or an execution time (in FIG. 3, it corresponds to “trigger condition/execution time”) to trigger an action, an action which tells the content of the action, and the time interval in which the action is taken, and the execution result of the action which tells a result of the action executed, in a row, corresponding each other. Here, stored are a standard action table information (FIG. 3 (a)) and an exception action table information (FIG. 3 (b), (c)).

Besides, in FIG. 3, the judgment table information defines each physical movement item by item, of an action corresponding to the execution result in the action table information. These action table information and judgment table information are determined based on the age, gender, the physical condition, the sick history, and etc. of a user of the S terminal 1. Referring the action table information stored in the memory part 14, the schedule execution management part 15 is able to execute action one by one according to trigger conditions or the execution time, and to execute an process corresponding to the execution result of the action, by referencing to the judgment table information.

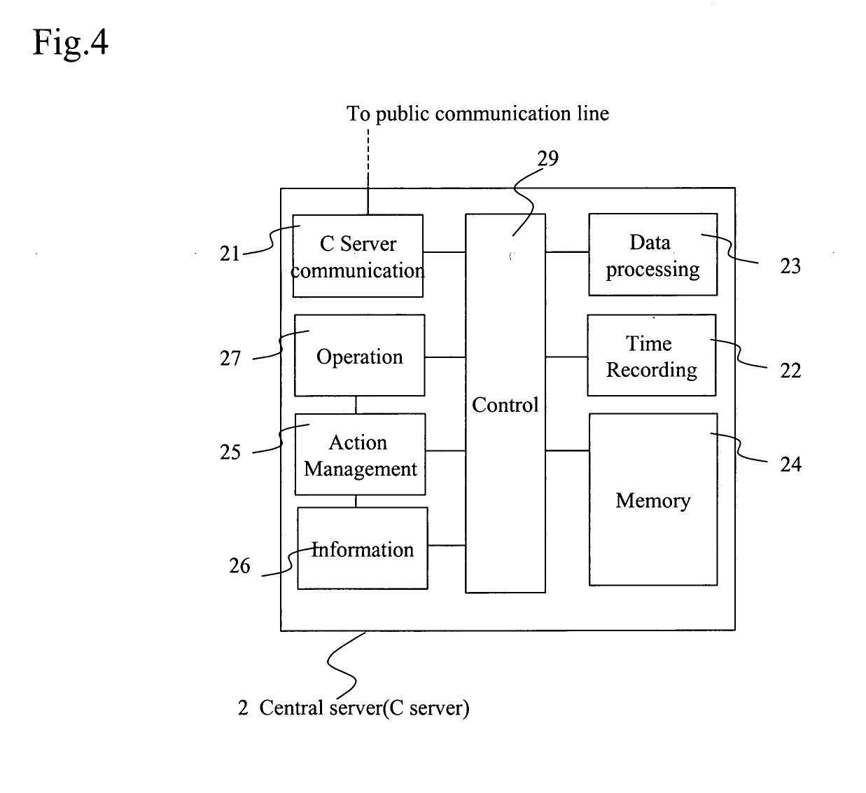

Next C Server 2 is explained. FIG. 4 is the outline block diagram showing the configuration of C Server 2. In this figure, the communication part 21 (noted as “C server communication”) communicates with the S Terminal 1, through the communication circuit 3. The time recording part 22 (noted as “Time Recording”) manages time based on the output from the internal clock circuit. Data-processing part 23 (noted as “Data processing”) processes various data. The memory part 24 (noted as “Memory”) stores not only the action table information and the judgment table information to be transmitted to S Terminal 1, but also the action table information and the judgment table information to be used inside of the C Server 2. The action table information and the judgment table information which are used inside of this C Server 2 will be explained later. Moreover, the memory part 24 stores various measurement results transmitted form S Terminal 1. The Action Management part 25 (noted as “Action Management”) execute action based on the action table information stored in the memory part 24, responding to the trigger condition and the execution time.

The information part 26 (noted as “Information”) is consist of a speaker and a liquid-crystal display, for example, and based on the direction of the control part 29, sends out an alarm sound, displays messages, and etc. Moreover, the information part 26 has a function to transmit a message to the portable terminal, the telephone, etc. carried by an operator. Operation part 27 (noted as “Operation”) is an input apparatus, such as a touch panel, a keyboard, and a mouse. The Control part 29 (noted as “Control”) transmits data between various parts in C server 2.

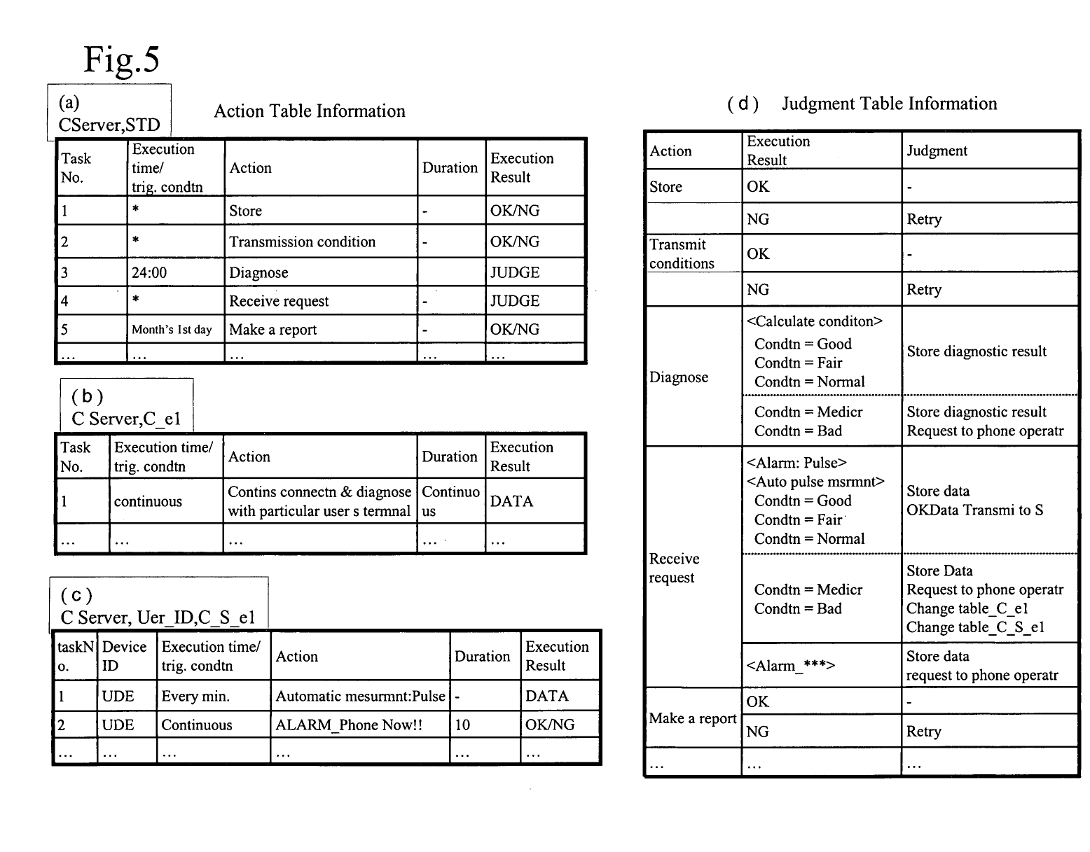

Next, information stored in the memory part 24 will be discussed. The FIG. 5 is to explain the action table information and the judgment table information stored in the memory part 24. In the figure, it is shown that the standard action table (FIG. 5a), the exception action table information (FIG. 5b), both of them to be used within C Server 2, and the exception action table information (FIG. 5c), and the judgment table information (FIG. 5d), both of them to be transmitted to the S Terminal 1. Referring the action table information stored in the memory part 24, the Action Management part 25 is able to perform action one by one according to trigger conditions or execution time, and to perform procession corresponding to the execution result of action, by referencing to the judgment table information. In FIG. 5, the action table information and the judgment table information, both of them to be used in the C server 2, and S Terminal 1. If there are multiple S Terminal 1's the action table information and the judgment table information to be transmitted to each S Terminal 1 are also stored.

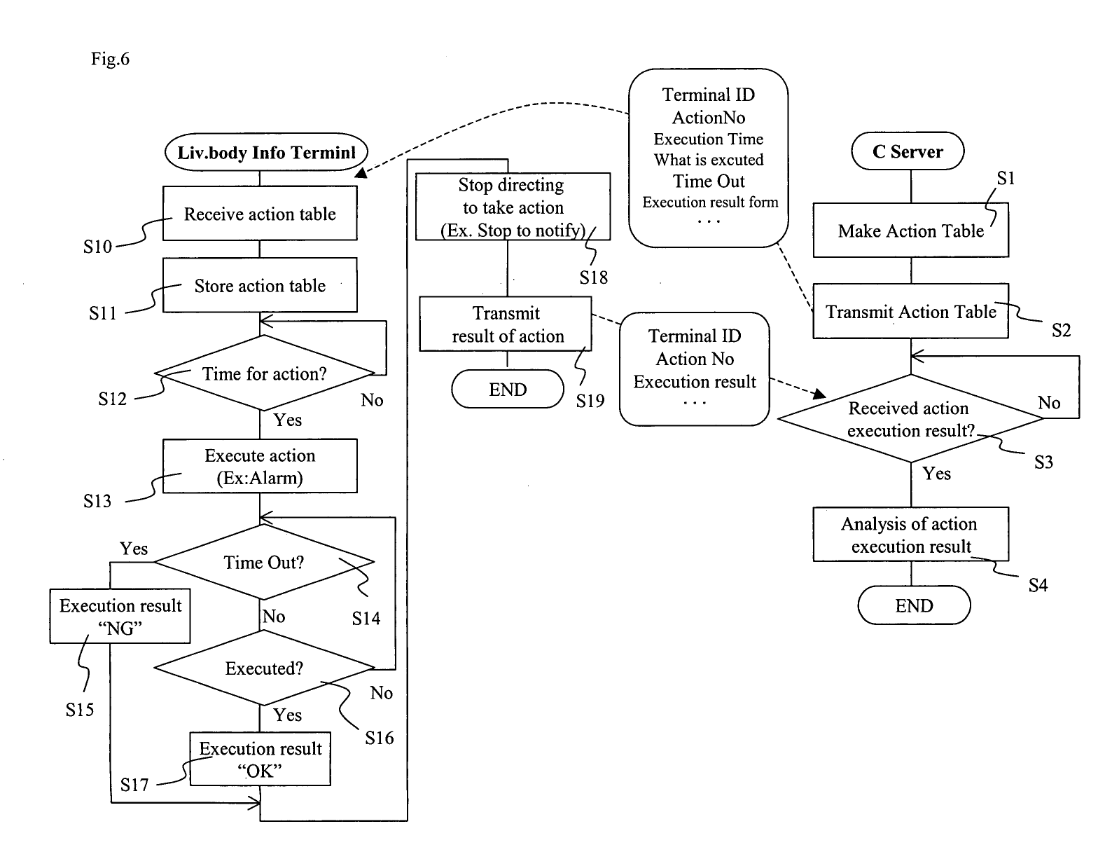

Next, operation in the said health-care system is explained using a drawing. FIG. 6 is a flow chart for explaining the outline of the operation of S Terminal 1 and C Server 2. An action table information corresponding to each user, is made by a doctor, etc. (Step S1). When the action table information is inputted by an operator or a doctor through the operation part 27 of C server 2, the control part stores information to the memory part 24. More specifically, the action table information, and the judgment table information to be transmitted to S Terminal 1, and the action table information and the judgment table information to be used in C Server 2 are created and stored in the memory part 24.

Next, the control part 29 reads the action table information and judgment table information stored in the memory part 24, sends the information to the S Terminal 1, through the communication part 11 (Step 2). The S Terminal 1 receives from C Server 2 through the communication part 11, the action table information, and the judgment table information (Step 10), and stores to the memory part 14 (Step 11). The Schedule Execution Management Part 15 of the S Terminal 1, detects whether the action table information has arrived at the execution time based on the time recorder 12 (Step 12). If it arrives at the execution time, it executes the action corresponding to the time (Step 13). Here, messages, such as “please take lunch”, are displayed on the display of the report part 16, and the alarm sound, at the same time, goes off.

The schedule execution management part 15 detects if the directed action is taken during the duration period, by detecting the input by the user of the result data of the notified action through the operation part 17 (Step S14). When the result data of action is not input within the duration time, the schedule execution management part 15 generates an execution result which shows the user not having carried out action (Step S15), stops the report part 16 to report, and to stop to direct the user to act (Step S18), and sends the execution result to C Server 2 (Step S19). When the fact that the action as directed was taken was input to the operation part 17 within the duration period (Step S16), the schedule execution management part 15 generates an execution result which shows the user took action as scheduled (Step S17), stops to direct to take action (Step S18), transmits the execution result of the action to the C Server 2 (Step S19). The C server 2, after receiving the execution result (Step S3), by referring to the judgment table information stored in the memory part 24, analyzes the execution result (Step S4).

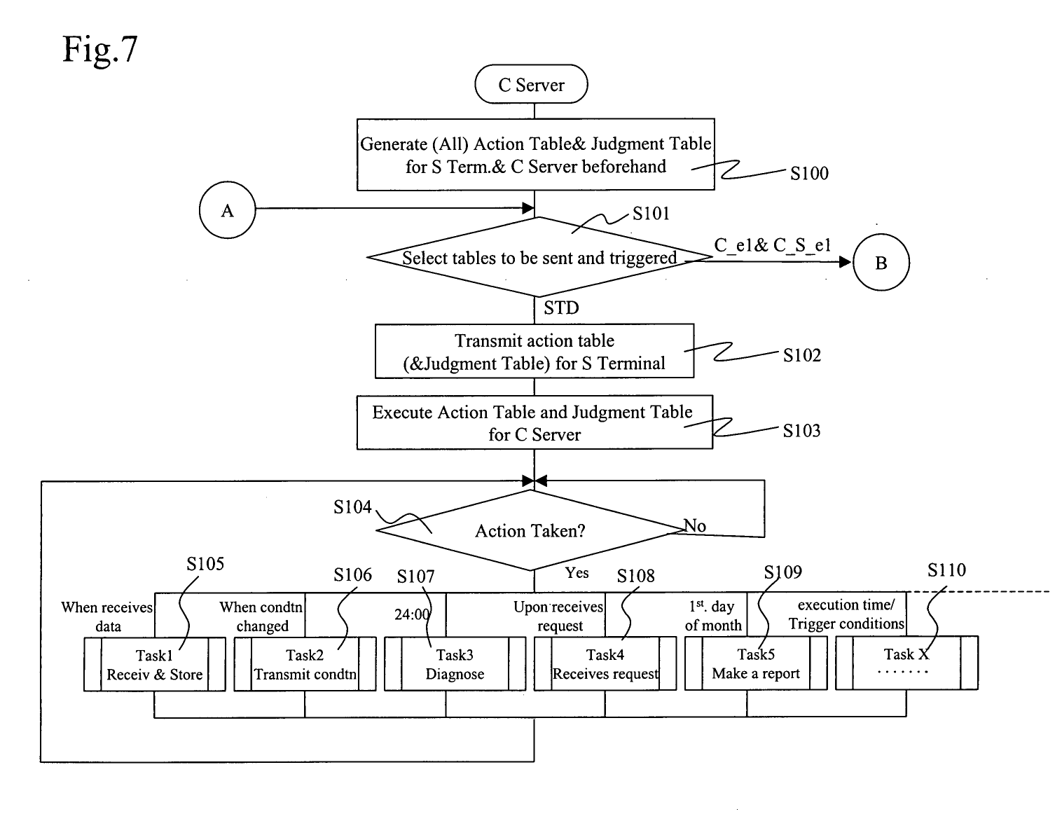

Next, other embodiment forms of operation in the said health-care system is explained. From FIG. 7 to FIG. 13 are flow charts to explain the operation of the C Server 2. In FIG. 7, after the operator of the C Server inputs information regarding action table information and judgment table information through the operation part 27, the control part 29 of the C Server 2 generates the action table information and the judgment table information for the S Terminal 1 and the C Server 2 (Step S100), and stores them to the memory part 24. The action management part 25 searches the action table information and the judgment table information of the S Terminal 1, using the terminal ID as the search key (Step S101), and sends the searched action table information and judgment table information to the S Terminal 1 through the communication part 21 (Step S102). First, the standard action table information and the judgment information are sent. And the action management part 25 reads the standard action table information and judgment table information for the C sever 2, and executes action according to the table action table information (Step S103). Here as an example, the standard action table, and the judgment table information, as in FIG. 5 are read.

Action Management part 25 detects whether action was taken or not, based on the trigger condition or the execution time in the standard action table, and the present time information outputted from the time record part 22 (Step S104). Here, the action Management part 25 will store the received data to the memory part 24 by executing action of Task one (FIG. 5) when it receives data (Step S105) from the S Terminal 1, in case the condition changed, will transmit the condition (Step S106) by executing action of task 2, if the time becomes at 24 hours, diagnose the user (Step S107) by executing the action of task 3 (FIG. 5), in case it receives a demand such as a judgment demand, receives the demand (Step S108) by executing the action of task 4 (FIG. 5), in case the time outputted from the time recorder 12 becomes first day of a month, it generates a report (Step S109) by executing the action of task 5 (FIG. 5). The action Management part 25 performs action of the task corresponding if such task is in the action table information and in the case, the time becomes the execution time of the task, or the condition meets with trigger conditions in the table (Step S110).

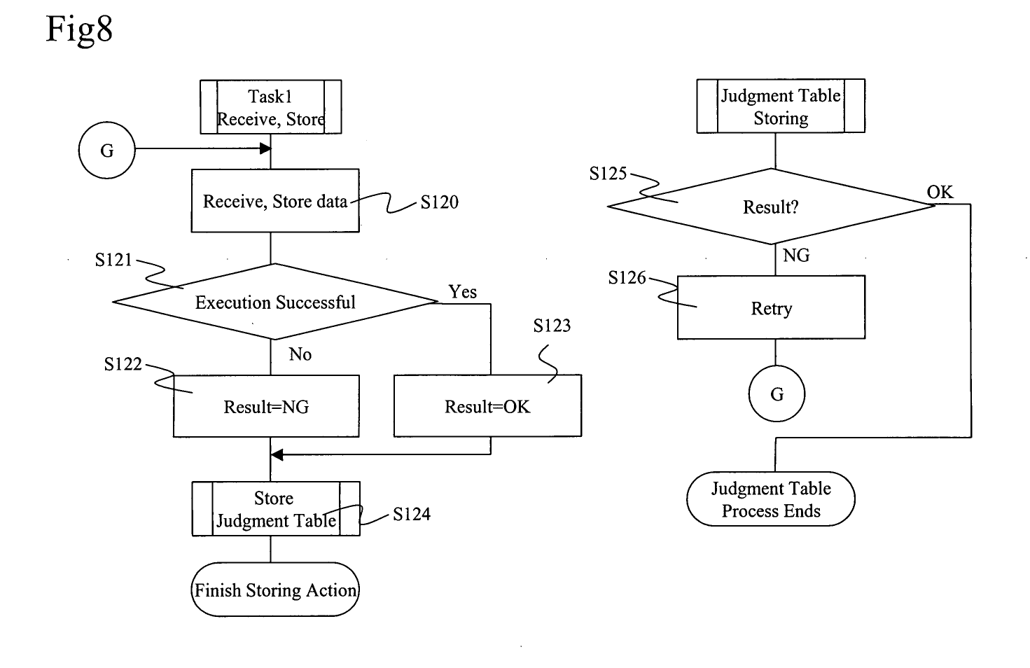

Next, each action in from step S105 to step S109, is explained using figures from FIG. 8 to FIG. 13. FIG. 8 is the flow chart to show the function of the C Server 2 in case the action is “receive data”. The Action Management part 25, when receiving data from the S Terminal 1, stores the data into the memory part 25 (Step S120), detects whether the reception of the data is successful or not (Step S121), and retain the detection result of “fault” (Step S122), or “success” (Step S123) temporarily as an execution result, and refers to the judgment table information for the required action (Step S124). The action management part 25, based on the execution result (Step S125), directs the S Terminal 1 to re-send the data in case the execution result is “fault” (Step S126), and moves to the step 120. On the other hand, if the execution result is “success”, the process ends.

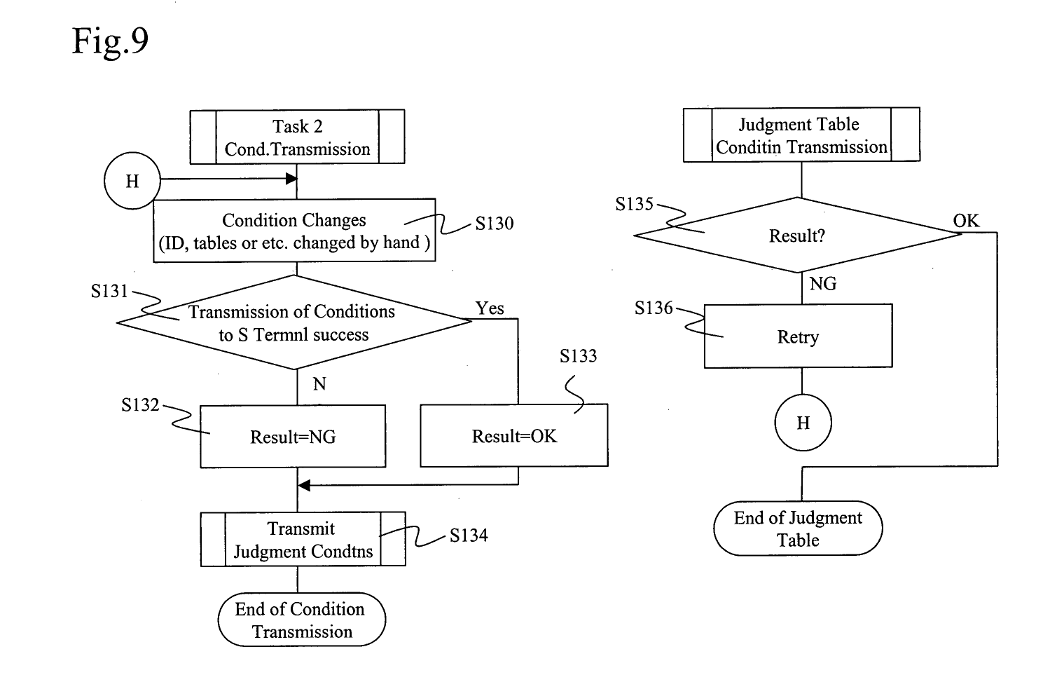

FIG. 9 is a flow chart to indicate the action taken by C Server 2 in case that the action in the action table information indicates condition transmission. The Action Management part 25, at changes of the ID of the S Terminal 1, the action table information, or the judgment table information (Step S130), transmits to the S Terminal 1, the action table information, and the judgment table information, and detects whether or not the transmission is successful (Step S131), stores temporarily the result of transmission of failure (Step S132) or success (Step S133) as an execution result and checks with the judgment table information corresponding to the action (Step 134). Based on the execution result (Step S135), in case of failure, the action management part 25 re-sends to the S Terminal 1, the action table information, and the judgment table information (Step S136), and moves to the step 130. On the other hand, if the execution result is “success”, this action is finished.

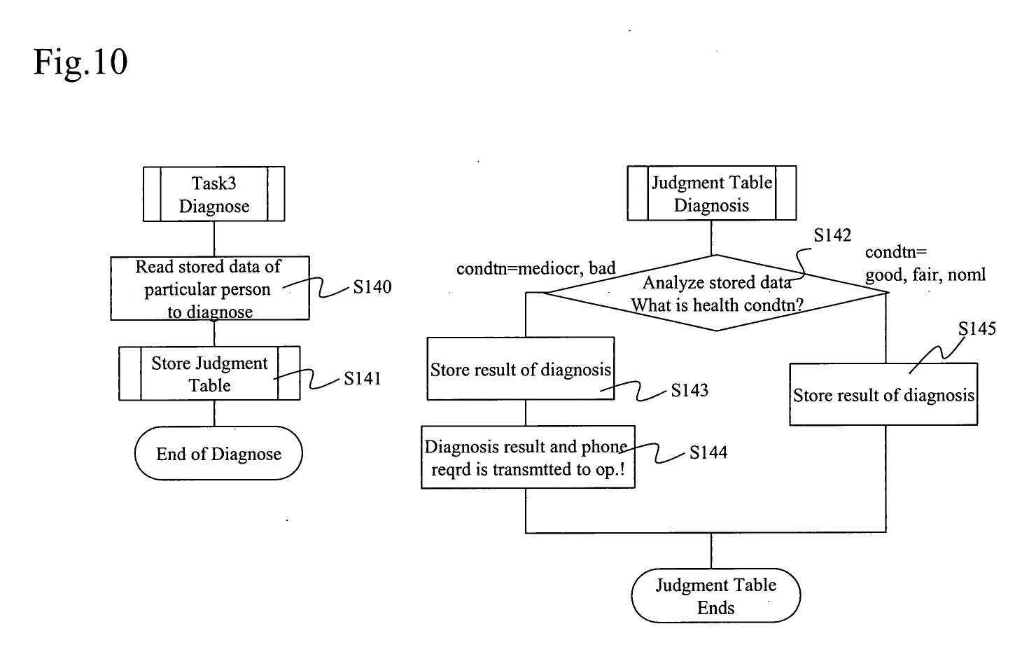

FIG. 10 is a flow chart to explain the process of the C Sever 2, in case the action is to diagnose a user. The Action Management part 25, when it detects that the time becomes at 24 hours, reads from the memory part 24, the stored data of the user to be diagnosed as an execution result (Step S140), refers to the judgment table information corresponding to this action (Step S141). And the action Management part 25, based on the judgment table information, analyzes the execution result, concerning the user's health condition (Step S142). The action Management part 25, when it detects the execution result corresponds to the codes of “mediocre or “bad” condition of the user, puts the result of the diagnosis “mediocre” or “bad” to the memory part 24 (Step S143). The reporting part 26 reports to the operator the result of diagnosis and that it is necessary to get in touch with the user to inform him/her that the treatment is required (Step S144). While if the action management part 25 detects that the execution result correspond to one of codes of “good”, “fair”, or “normal” state of the user, one of the detected results of diagnosis of “good”, “fair”, and “normal” is stored in the memory part 24 (Step S145).

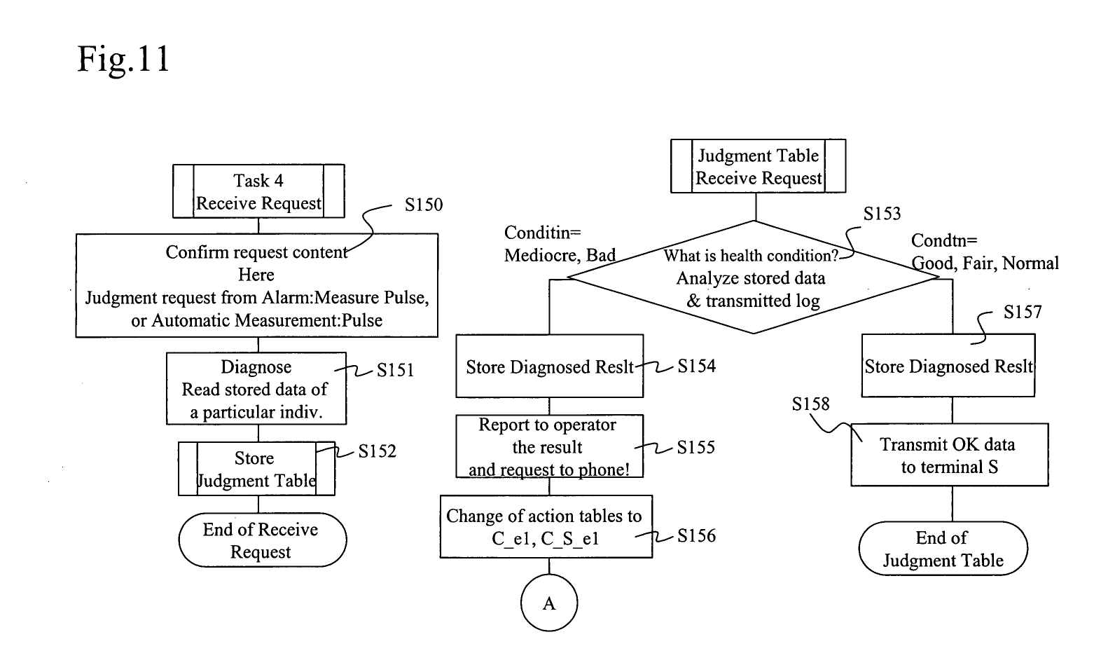

FIG. 11 is a flow chart to explain the process taken by C Server 2, when it receives an action request. The action management part 25, upon receiving a judgment request from the S Terminal 1 (Step 150), after receiving data which arrives with the judgment request, reads the stored data of this user from the memory part 24 (Step S151), and refers the judgment table information corresponding to this action (Step S152). The receiving data and the stored data of this user read from the memory part 24, correspond to execution result. The action management part 25 analyzes data sent from the S Terminal 1 and the stored data, or the execution result, concerning the health condition of the user, based on the judgment table information (Step S153). In case that the action management part 25 detects that use's condition is the one corresponding to the code, “mediocre”, or “bad”, based on the execution result, the result of diagnosis “mediocre” or “bad” is stored in the memory part 24 (Step S154), and the report part reports the diagnosis result to an operator (Step S155). The action table information, now in use, is changed from the standard action table information to the exceptional action table information (example. FIG. 5,(b),(c)) (Step S156).

The action Management part 25, on the other hand, in case as the result of analysis, user's condition is detected as the one corresponds to one of codes, “good”, “fair”, or “normal”, stores the analysis result, “good”, “fair”, or “normal”, to the memory part 24. The communication part 21, sends the result to the S Terminal 1 to inform that “the result was good” (Step S158).

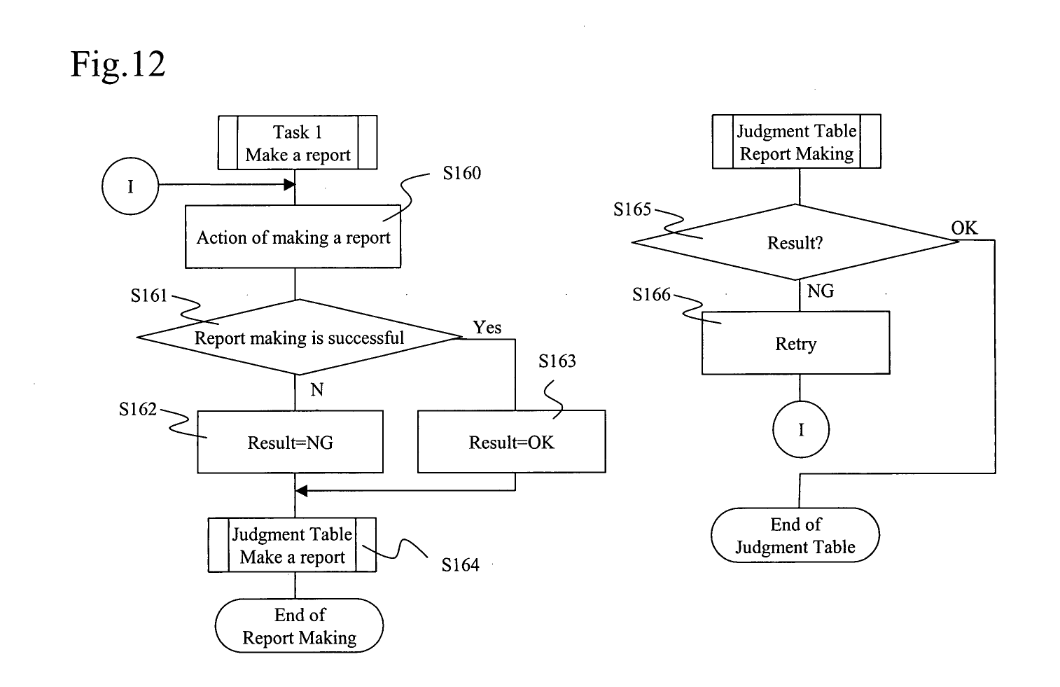

FIG. 12 is a flow chart to explain the process taken by the C Server 2, in case the action is to create an report. The action management part 25 creates a repot on the first day of the month (Step S160). This report creation is a process in which the information is created for managing a user's healthy condition. Upon the detection of whether the creation was success or not (Step S161), the action Management part 25 creates data of “failure” or “success” of the creation as an execution result, based on the detection (Steps S162, S163), and refers to the action table information corresponding to this action (Step S164). Next the action management part 25, based on the execution result and the judgment table information (Step S165), in case the execution result was “failure” (Step S166), moves to the process to the step S160, for re-try, and in case the execution result was “success”, stores the created report in the memory part 24.

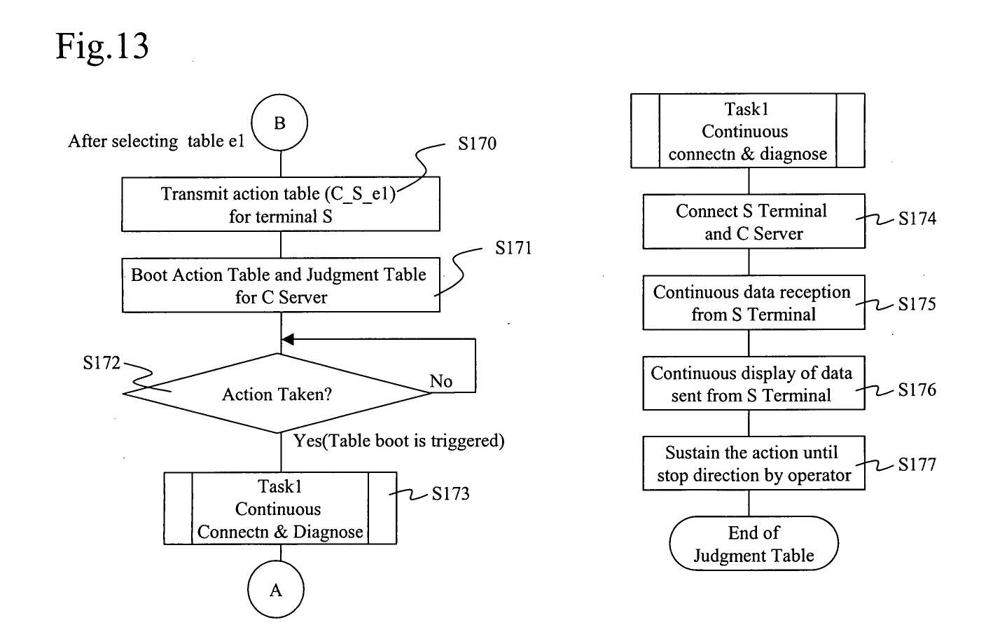

FIG. 13 is a flow chart to explain the process taken by the C Server 2, in the case that the action table information is changed to the exception action table information from the standard action table information. This process corresponds to the above-mentioned step S156 and subsequent processes. The Action Management part 25 in the table selection of Step S101, after selecting the exception action table information for the S Terminal 1 and the exception action table information for the C Server 2, transmits the exceptional action table information to the S Terminal 1, by the communication part 21 (Step S170), boots and executes the exception action table information and the judgment table information for the C Server 2 (Step S171). The action is executed at the trigger condition or at the execution time in the exception action table information (Step S172). Here, executed is the task 1 of continuous connection and continuous diagnosis, as illustrated for example in FIG. 5 (b) (Step S173).

The action management part 25, based on the action of this task 1, connects the communication line between S Terminal 1 and C Server 2 (Step S174), based on the exception action table information sent from the S Terminal 1 in the step S170, receives data continuously transmitted from the S Terminal 1 (Step S175), and displays the received data on display device etc. one by one (Step S176). This action is continued until directions of an end are inputted from an operator of the C Server 2 (Step S177). When it ends, the process moves to Step S101.

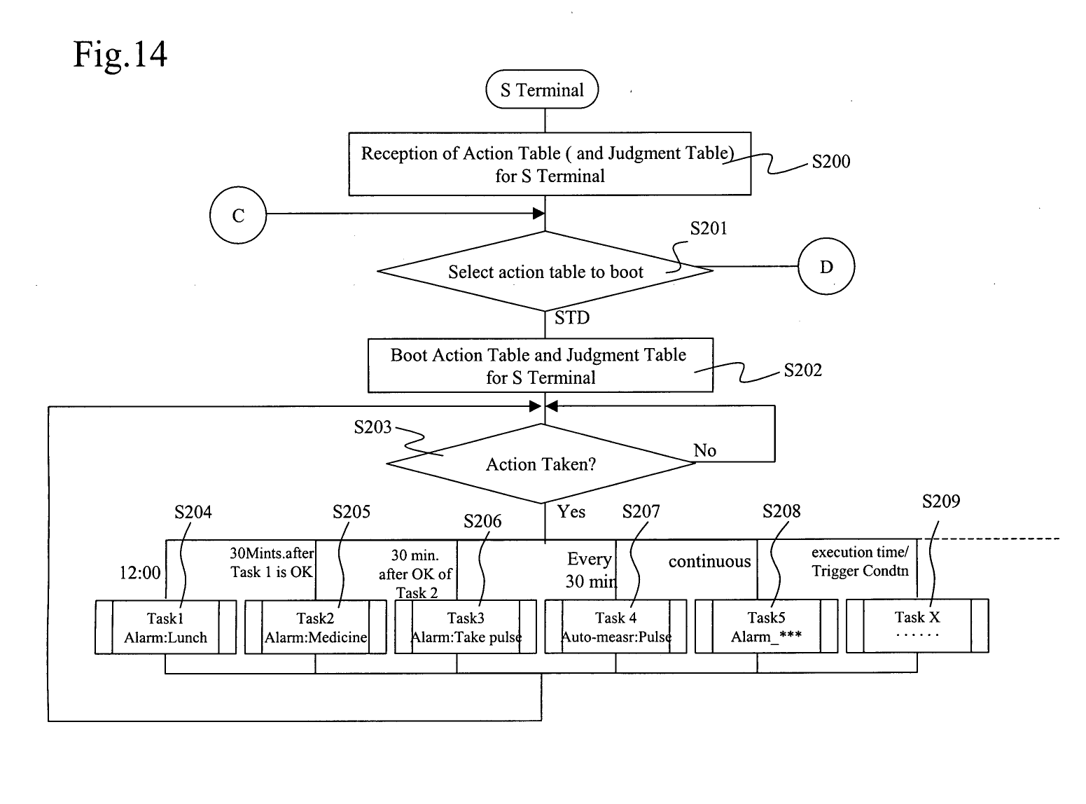

Next, processes the S Terminal 1 of other embodiment in the said health-care system is explained. From FIG. 14 to FIG. 19 are flow charts to illustrate the process of the S Terminal 1. First, in the FIG. 14, the control part 19 of the S Terminal 1 receives the action table information and the judgment table information for the S Terminal 1 (Step S200), and referring to the table identification codes designated to the received action table information, selects the action table information to boot (Step S201). In this case, since the action table information sent from the C Server 2 is the standard action table information, the control part 19 directs the schedule execution management part 15 to boot this standard action table information, and the judgment table information (Step S202). The case in which the exception action table is selected (as the result of change of the action table information), will be explained. The schedule execution management part 15, based on the direction from the control part 19, executes action, following the standard action table information. Here, for example as in FIG. 3, the standard action table information, FIG. 3(a), and the judgment table information, FIG. 3(d) are read and executed.

The schedule execution management part 15, based on present time information outputted from the time recorder 12, and the trigger condition or the execution time in the standard action table information, detects if the action is to be executed or not (Step S203). Here, the schedule execution management part 15, when the time is the execution time “12 hours”, executes the action of Task 1 (FIG. 3), thus gives lunch alarm (Step S204), and after 30 minutes past after the normal completion of task 1, by executing task 2 (FIG. 3) and gives off the alarm to take medicine (Step S205), and after 30 minutes of the normal completion of the task 2, by executing the action of task 3 (FIG. 3), gives off alarm to take pulses (Step S206), and at every 30 minutes, by executing the action of task 4 (FIG. 3), takes pulses (Step S207), and if the action is continuous predetermined movement, executes the action continuously of the task 5 (Step S208). The schedule execution management part 15, at other execution time or trigger condition, executes the action corresponding to that task (Step S209).

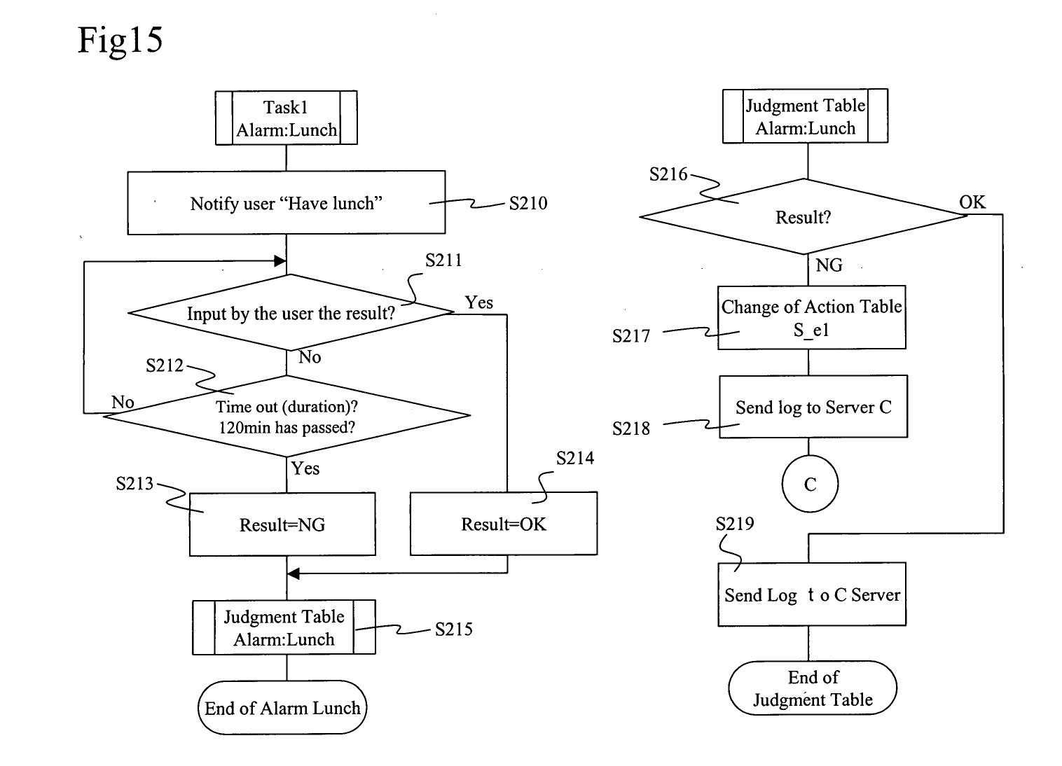

Next, each action at from Step S204 to Step S207 is explained using figures from FIG. 15 to FIG. 19. FIG. 15 is a flow chart to explain the process taken by the S Terminal 1, when it gives off alarm for lunch. The schedule execution management part 15, when the time is at 12 o'clock, according to the content defined by the action in the standard action table information, displays the message “Please have lunch” through a display device of the report part 16, and at the same time, gives off sound alarm (Step S210, Task 1 of the standard action table information for the S Terminal 1). The schedule execution management part 15 detects whether the user had lunch by data inputted through the operation part 17 (Step S211).

The schedule execution management part 15, in the case data is not inputted, either gives off alarm sound or displays the message “Please have lunch”, and detects whether the continuation time (120 minutes) has passed or not (Step S212). In the case the result data is not inputted even if the continuation time passed, it stores the result data “Action NG” temporarily as an execution result which indicates that the user did not take action as directed (Step S213).

On the other hand, in Step S211, if the user had lunch, and the result data indicating that lunch was taken was inputted, the schedule execution management part 15 holds the result data “Action OK” temporarily as an execution result (Step S214), and refers the judgment table information corresponding to this action (Step S215). The schedule execution management part 15, refereeing to the temporary hold the execution result (Step S216), if the execution result is “Action NG”, the action table information is replaced by the exception action table information (FIG. 3(b)) (Step S217), and transmits the log record that the execution result is “Action NG” and the schedule is changed to the exception action table, to C Server 2 (Step S218). The process now moves to Step S201, of FIG. 14. On the other hand, if the execution result is “Action OK”, the schedule management part 15 sends the log which indicate that the execution result is “Action OK” to C Server 2 (Step S219). Thereby, in C Server 2, the log is stored (Task 1 in the standard action table information for the C Server 2).

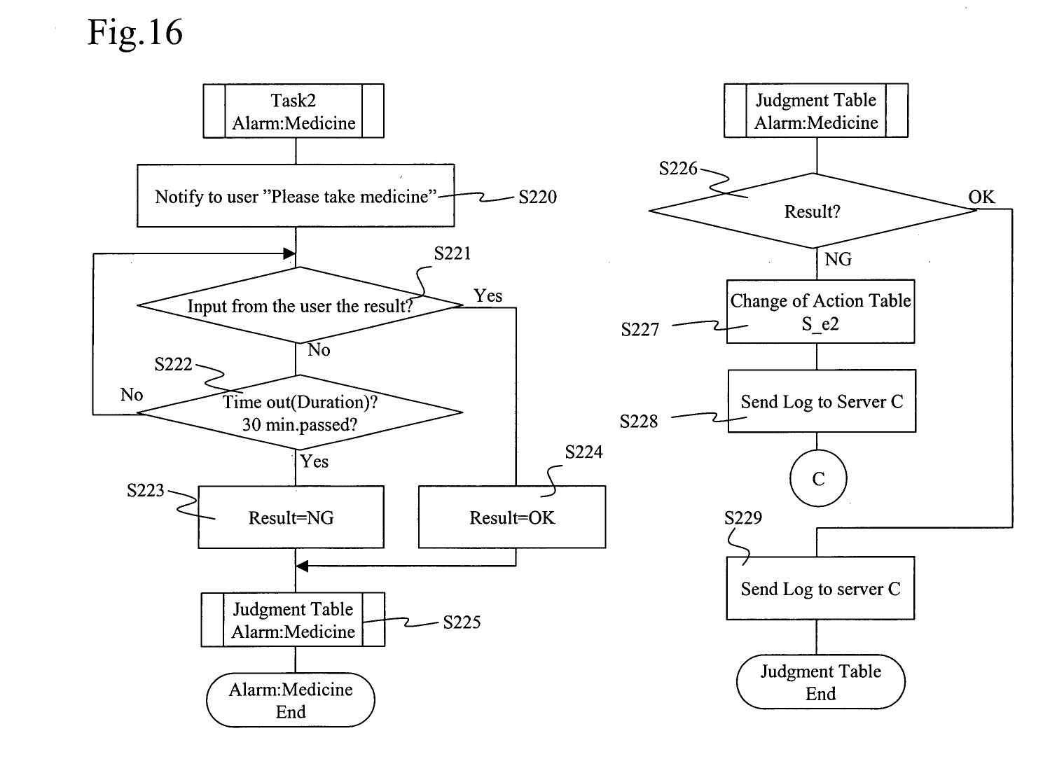

FIG. 16 is a flow chart to explain the process taken by the S Terminal 1, when it alarms for medication. The schedule execution management part 15, following the content defined in the action of the standard action table information, displays the message “Please take medicine”, by the display part, releases an alarm sound (Step S220), and detects whether or not the result data indicating that the user took medicine is inputted through the operation part 17 (Step S221). The schedule execution management part 15, when data is not inputted, after following this action, detects whether the duration time (30 minutes) has passed or not (Step S222), and in the case the result data is not inputted after the duration time, temporarily stores the result data “Action NG” as an execution result that indicates the user did not follow as directed in the action (Step S223), and in the case the result data is inputted, stores “action OK” as an execution result that indicates the user followed as directed in the action (Step S224), and references to the judgment table information corresponding to this action (Step S225). And the schedule execution management part 15, referring to the temporarily hold execution result (Step S226), if the execution result is “Action NG”, it replaces the action table information with the exception action table information (FIG. 3 (c)) (Step S227), and sends log to C Server 2 which says that the execution result is “Action NG”, and the action table information is exchanged with the exception action table information (Step S228). Then process moves to the Step S201 of FIG. 14. In the case that the execution result is “Action OK” on the other hand, the schedule management part sends the log to the C Sever 2 which indicates the execution result is “Action OK” (Step S229). In addition, the action execution management part 15 may continuously display, or give off alarm sound of the message, “Please take medicine.” until the continuation time passes, or until the result data from a user is inputted, or until the user inputs a confirmation of having checked the contents.

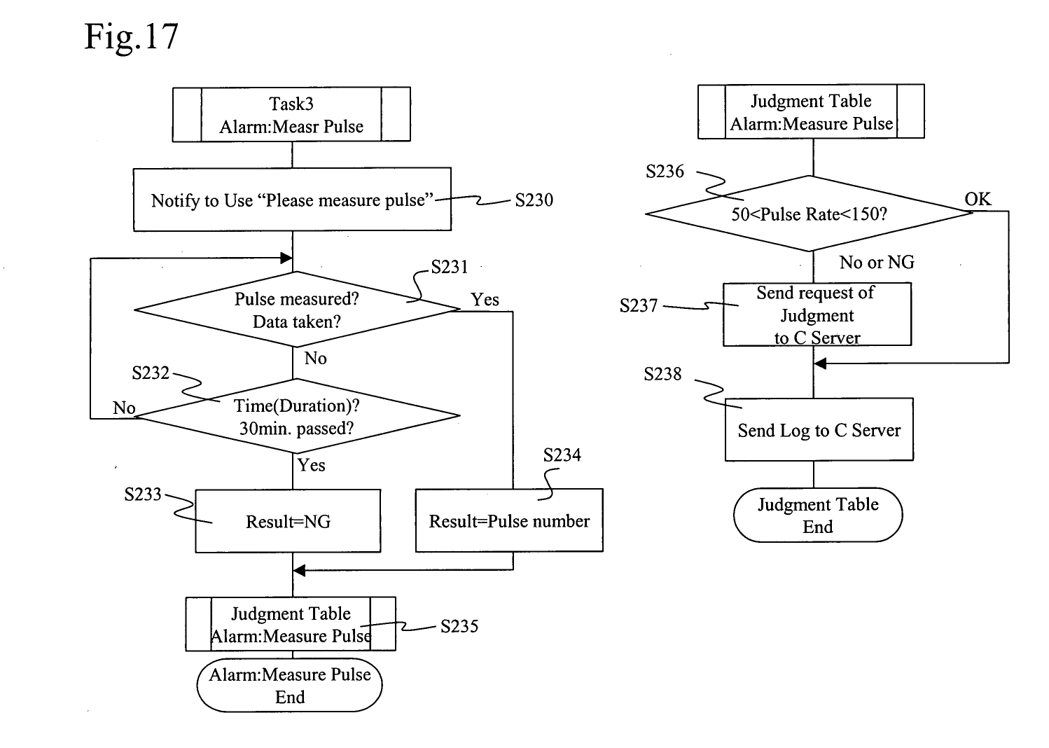

FIG. 17 is a flow chart of the process taken by the S Terminal 1 in the case of performing the pulse measurement. The schedule execution part 15, following the content of the action defined in the standard action table information, displays the message “Please measure the pulse” at the display device of the report part 16, or gives off an alarm sound (Step S230). And the schedule execution management part 15 detects if the pulse measurement is made by measurement part 13, following the direction for the pulse measurement, through the operation part 17 inputted by the user (Step S231). The schedule execution management part 15, in the case that the measurement was not made, or the result data as the measuring result was not inputted, detects whether or not the duration time (30 minutes) has passed (Step S232) before this action was performed, and if the result data was not inputted after the duration time, the result data “Action NG” which indicates that the user did not measure the pulse, is hold temporarily as an execution result (Step S233), and in Step S231, either measurement was made or pulse measurement result was inputted through operation part 17, as the result data, the measurement data is hold temporarily as an execution result (Step 234) references to the judgment table information corresponding to this action (Step S235). And the schedule execution management part 15 detects if the pulse is between 50 and 150, by referencing to the temporarily hold execution result (Step S236). The schedule execution management part 15, in the case that the pulse rate is not between 50 to 150 bps, or in the case of “Action NG”, sends a judgment request to the C Server 2 (Step S237), and at the same time sends to the C Server 2 the measurement data of pulses and the message “Action NG” as the log record. On the other hand, if the pulse rate is between 50 to 150 bps, the schedule execution management part 15 sends to the C Server 2 the log to indicate the measured data of pulses and the pulse is normal (Step S238). In addition, in this embodiment of the invention, when the pulse rate is between 50 and 150 bps, the pulse is defined normal, while the criteria of the pulse being normal is not limited to this criteria, rather, it can be defined different ways based on the user's condition, healthy condition, and etc.

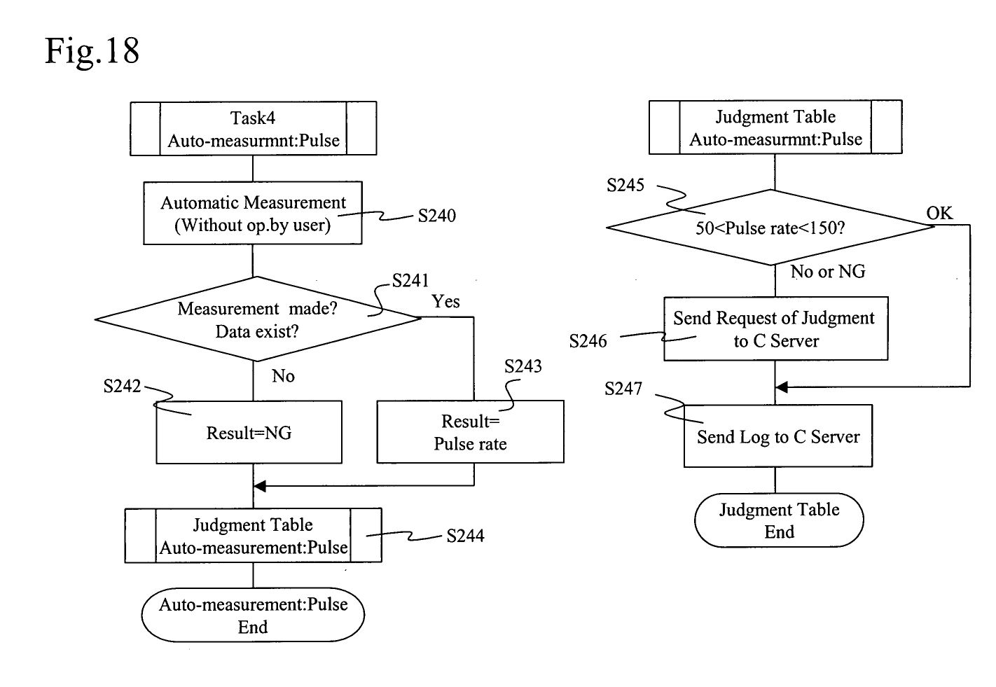

FIG. 18 is a flow chart to explain the process taken by the S Terminal 1 in the case of automatic pulse measurement. The schedule execution management 15 following the content defined in the action of the standard action table information, measures pulse automatically (Step S240). The automatic measuring, here, mean that without the direction from the user, the schedule execution management part 15, at the time specified in the action table information, measures the pulse by the measurement part 13. The schedule execution management part 15 detects whether or not, at the prefixed time, measurement was performed of the pulse by the measurement part 13 (Step S241). The schedule execution management part 15, when the measurement was not performed, of the measurement result was not stored in a memory, temporarily holds the result data “Action NG” as an execution result which expresses that the pulse measurement was not made (Step S242), while in the Step 241, if the pulse measurement was performed normally, holds the measured data temporarily as an execution result (Step S243), and refers to the judgment table information corresponding to this action (Step S244). And the schedule execution management part 15 detects if the pulse rate is between 50 and 150 bps, by referring to the temporary hold execution result (Step S245). In the case that in the detected result, pulse rate is not between 50 and 150 bps or the case is “Action NG”, the schedule execution management part 15 sends to C Server 2 judgment request (Step S246), and at the same time, sends the pulse measurement data, or “Action NG” as a log (Step S246). On the one hand, in the case the pulse rate is between 50 and 150 bps, the schedule execution management part 15 sends to the C Server 2 the pulse measurement data and the log which shows the pulse was normal (Step S247).

About the action of Task 5 in the Step S208, similarly as in flow charts, from FIG. 15 to FIG. 17, the schedule execution management part 15, reports to the user, through the report part 16, monitoring the duration period. The schedule execution management part 15 sends to C Server 2, the result data when the result data is inputted by the user within the duration period, or the fact that the user did not take action as required if the user did not input the result data corresponding to the action within the duration period. Moreover, in this embodiment, it detects that the pulse is normal when the pulse rate is between 50 and 150 bps, the criteria of the pulse's normality is not limited to this case, and the criteria may appropriately determined upon a user's physical condition, healthy condition, and etc.

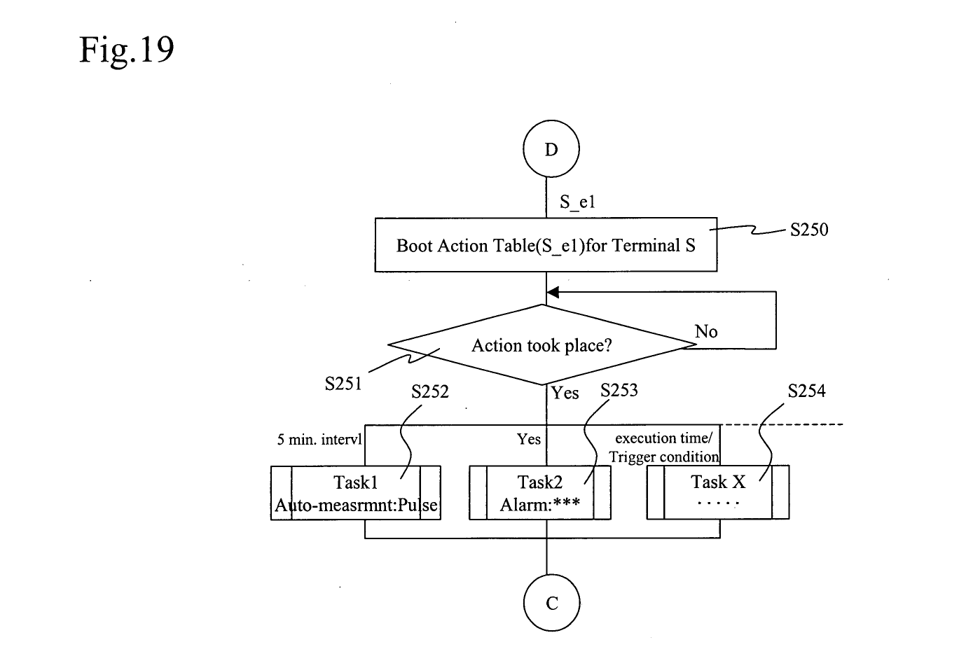

Next, the case in which the exception action table information is started is explained using the flow chart FIG. 19. The schedule execution management part 15, at selecting a table to start, at the Step 201 of FIG. 14, if it is directed to start the exception action table information, starts the exception action table information which is already directed to start, by changing the action table information (Step S250). In the case that the action table information is directed to be changed at Task 1, the exception action table depicted by FIG. 3(b) is started, and in the case that the action table information is directed to be changed at Task 2, the exception action table depicted by FIG. 3(c) is started, and in the case that the schedule execution management part receives the exception action table information in FIG. 11 step S156, the exception action table in FIG. 5(c) is started. Here, the case where the exception action table information shown in FIG. 3(b) is started, is explained.

When the exception action table information is started, the schedule execution management part 15, following the exception action table information, detects whether to generate an action, based the present time information outputted from the time recorder 12, and the trigger condition or the execution time in the standard action table information (Step S251). In this embodiment, when time becomes the execution time, the action “Automatic measurement of pulse” of Task 1 is performed (Step S252). The actions of this task 1 are repeated in a predetermined time interval (5 minutes). At the start up of the exception action table information, the schedule execution management part 15 reports through the report part 16, alarm “Please take lunch” as specified in the Task 2 (Step S253). In addition, when other tasks exist in the exception action table information, the action is performed according to the trigger condition or the execution time specified in the action (Step S254)

More specifically, the schedule execution management part 15, following to the exception action table information (FIG. 3(b)), automatically measures pulse at every five minutes by the measurement part 13 (Task 1 FIG. 3(b) of the exceptional action table information for S Terminal 1), and at the same time continuously tells to take a meal. And the schedule execution Management part 15, detects whether the measured pulse rate which is the execution result is within the normal values, based on the judgment table information, and if the pulse rate is between 50 and 150 bps, sends the log to the C Server 2. When the pulse rate is not between 50 and 150 bps, the schedule execution management part 15 sends the log which shows that it is outside of the normal range, and request C Server 2 for judgment. Receiving the request, the C Server 2 outputs “JUDGE” corresponding to an execution result to judgment table information following the Task 4 in the standard action table information, and judges the condition of the user. When the judgment result is “mediocre” or “bad”, then the data is stored, and urges the operator to make a phone call to the user by display the request data on the display device, and replaces the action table information with the exception action table information. The same process can be applied to taking medicine or measuring the pulse. When the action defined in the exception action table information is completed, the action table information is exchanged with the standard action table information. In addition, in this embodiment of the invention, when the pulse rate is between 50 and 150 bps, the pulse is defined normal, while the criteria of the pulse being normal is not limited to this criteria, rather, it can be defined different ways based on the user's physical condition, healthy condition, and etc. And in this embodiment, the case where the standard action table information and the judgment table information are transmitted form C Server 2 to S Terminal 1 is explained, the exception action table information as well as the standard action table information and the judgment table information may be transmitted to the S Terminal 1. In this case, the communication line to transmit from the C Server 2 to the S Terminal 1 is cut off once. After this, even if “Action NG” occurs, the S Terminal 1 performs the action based on the exception action table information which is already received and stored, and if necessity arises, restores the communication, and transmits various data. In this way, even if “action NG” occurs when the communication is interrupted, and it becomes necessary to start the exceptional table information, it is possible to respond immediately without waiting for the restoration of the communication.

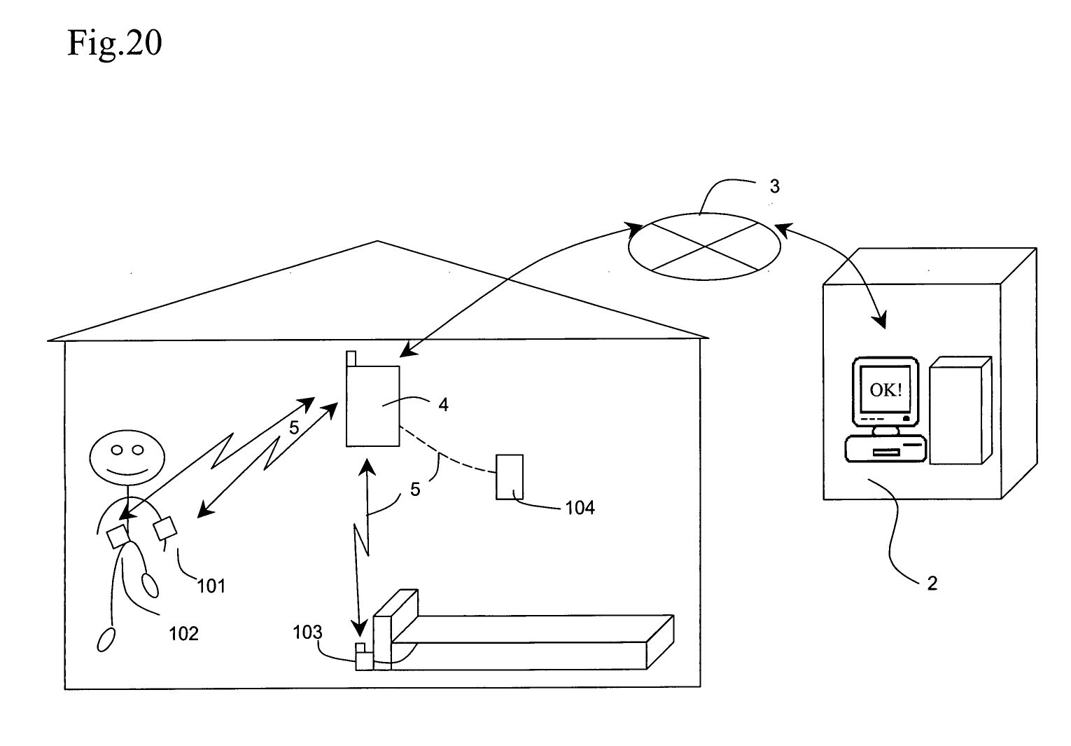

FIG. 20 depicts the outline composition of the health-care system in the second embodiment of present invention. In this figure, parts corresponding to the ones in FIG. 1 are marked by the same mark, and the explanation is omitted. In the figure, the C Server 2, as an example of the health-care device is connected to a personal sever 4 (hereafter called P Server 4) by the communication line 3. P Server 4 communicates with the wrist watch type terminal 101, the portable type terminal 102, the sensor terminal 103, and the desk top type terminal 104. The wrist watch type terminal 101 has the same composition as the said S Terminal 1, thus the explanation of the terminal is omitted. The portable type terminal 102 is attached to the body of a user, and it has the function of measuring motions (acceleration, angular velocity, etc.) of the user at the wearing position. The sensor terminal 103 measures the breathing, heart beat, body movement, snoring, and etc. of a user when he/she is sleeping on bed. The desk top type terminal 104 measures glucose in the blood and etc. are measured of a user. The P server 4 could be a general computer, and could be installed at each home.

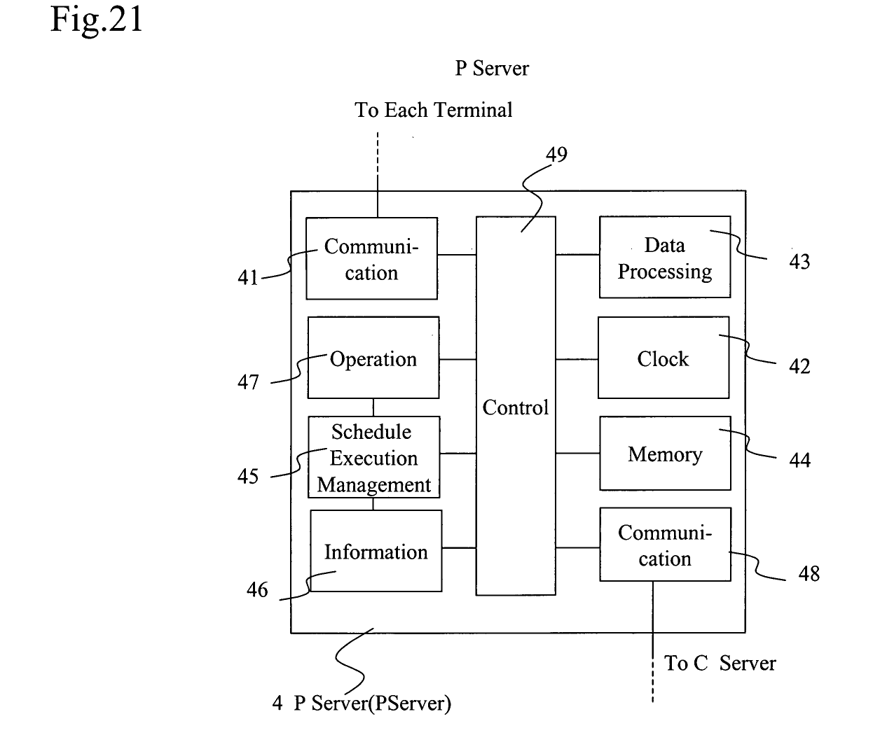

Next P Server 4 is explained further. FIG. 21 is an outline block diagram for explaining composition of the P Server 4. In this figure, the communications part 41 communicates with each living-body information terminal (wrist watch type terminal 101, portable type terminal 102, sensor terminal 103, desk top type terminal 104). The time record part 42 manages time based on the output from the internal clock circuit. The data processing part 43 processes various data. The memory part 44 stores the action table information and the judgment table information to transmit to each living-body information terminal, and at the same time, stores the action table information and the judgment table information for the use at the P Server 4 as well as the action table information and the judgment table information for the use at each living-body information terminal.

The execution management part 45 performs the action corresponding to the trigger condition or the execution time based on the action table information stored in the memory part 44. The information part 46 comprises a speaker, and the display device such as the liquid crystal display device and etc. and following the direction of the control part 49, gives off alarm sounds, displays messages and others. The operation part 47 is an input/output device such as a touch panel, or a key board, or a mouse. The communication part 48 communicates with the C Server 2. The control part 49 transfers data between the P Server 4 and each part.

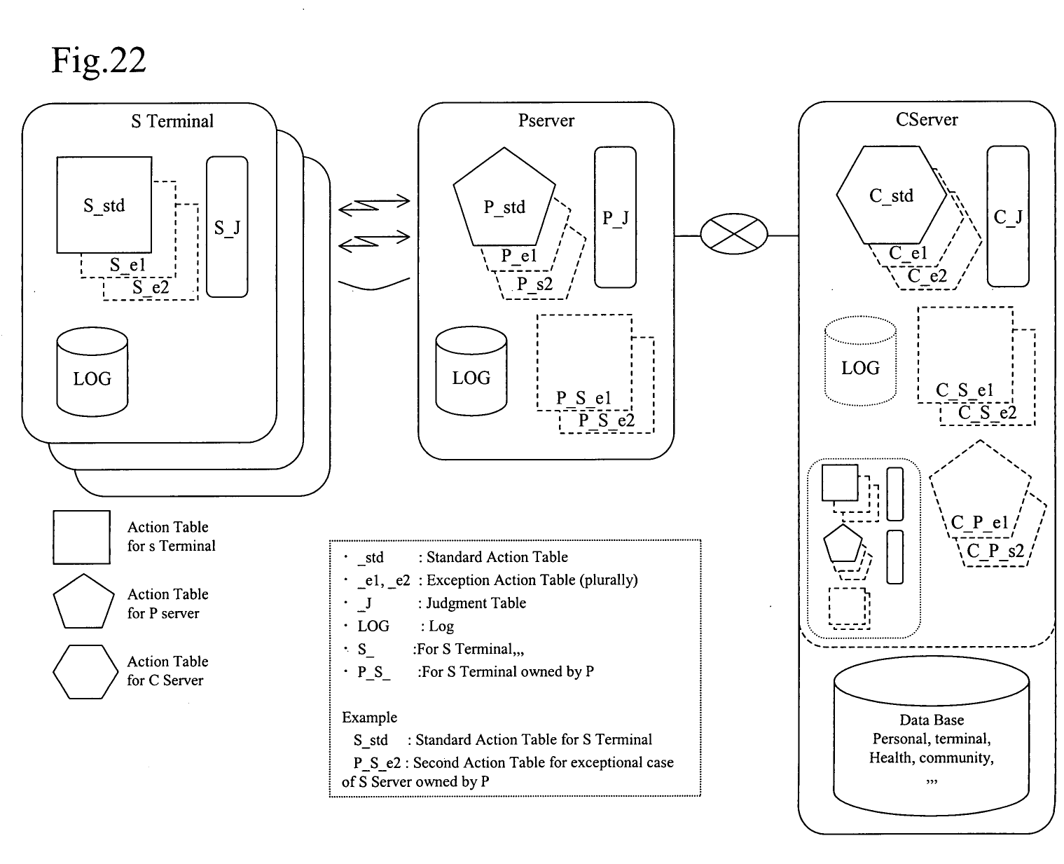

FIG. 22 is a drawing for explaining the state of how the action table information, and the judgment table information are stored. In this figure, all action table information and judgment table information are generated and stored in C Server 2. Action table information and judgment table information generated in the C Server 2 may be stored also at each living-body information terminal (S Terminal 1), P server 4, and C Server 2. Or it may be that only stored in C Server 2, and at each action, information is transmitted to each living-body information terminal (S Terminal 1 and etc.) and a P Server 2 where the action is performed.

In the example depicted in FIG. 22, action table information and judgment table information of its own use and such information used by the server and terminals branched out from it are stored. Viz. The C Server 2 stores the action table information and judgment table information for itself; for P server 4, and for each living-body information terminal. The P Server 4 stores action table information and judgment table information for P Server 4, and for each living-body information terminal. Each living-body information terminal stores the action table information and judgment table information for its own use.

By storing the action table information and judgment table information for its own and branched out servers and terminal (P Server 4 and each living-body information terminal), it is possible to up-date the action table information and the judgment table information for its own use and for branched out terminals and servers. For example, C Server 2 has a function to change the action table information and judgment table information of C Server 2 itself, P Server 4, and each living-body information terminal. P Server 4 has a function to updates the action table information and judgment table information of itself and its subordinate each living-body information terminal. And each living-body information terminal has a function of update its own of action table information and judgment table information. Whichever action table is updated, the update information (User ID, Terminal ID, Table ID, Time information and a log) is transmitted to C Server 2 and stored therein as a log.

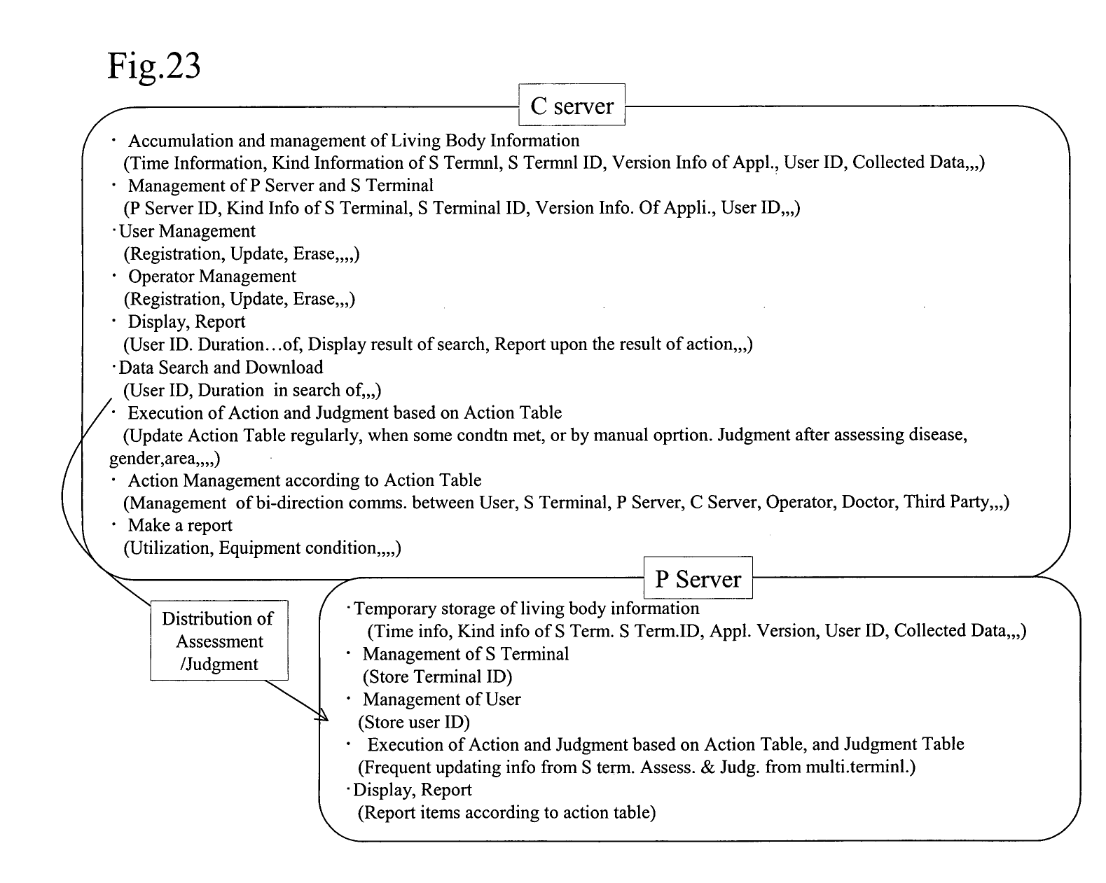

Next, using FIG. 23, distribution of functions between C Server 2 and P Server 4 is explained. C Server 2 has functions depicted in the upper row in FIG. 23, whereas P Server 4 has functions depicted in the lower row in FIG. 23. Here, judgment items using the action table information and the judgment table information are distributed between the P Sever 4 and the C Server 2. In the P server 4, the judgment table information is created to comprehensively review data of the user which incorporates measurement result transmitted from the living-body information terminal, and the result data inputted by the user. The judgment is made using this judgment table information. In the C server 2, the judgment table information is created which includes data not only of one user but also such information received from each P server 4, as well as data created by coordinating database of other systems to comprehensively review data by kind of diseases, by gender, and by locality. The judgment is made using this judgment table information.

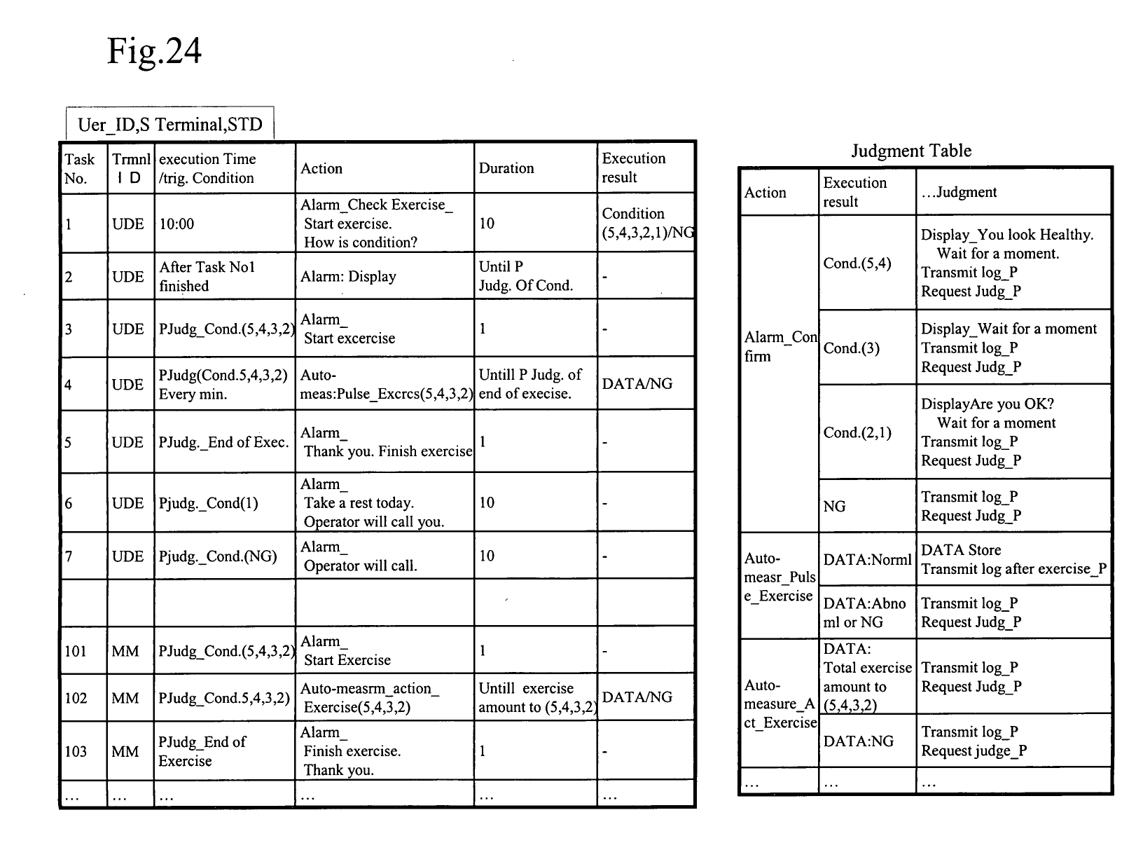

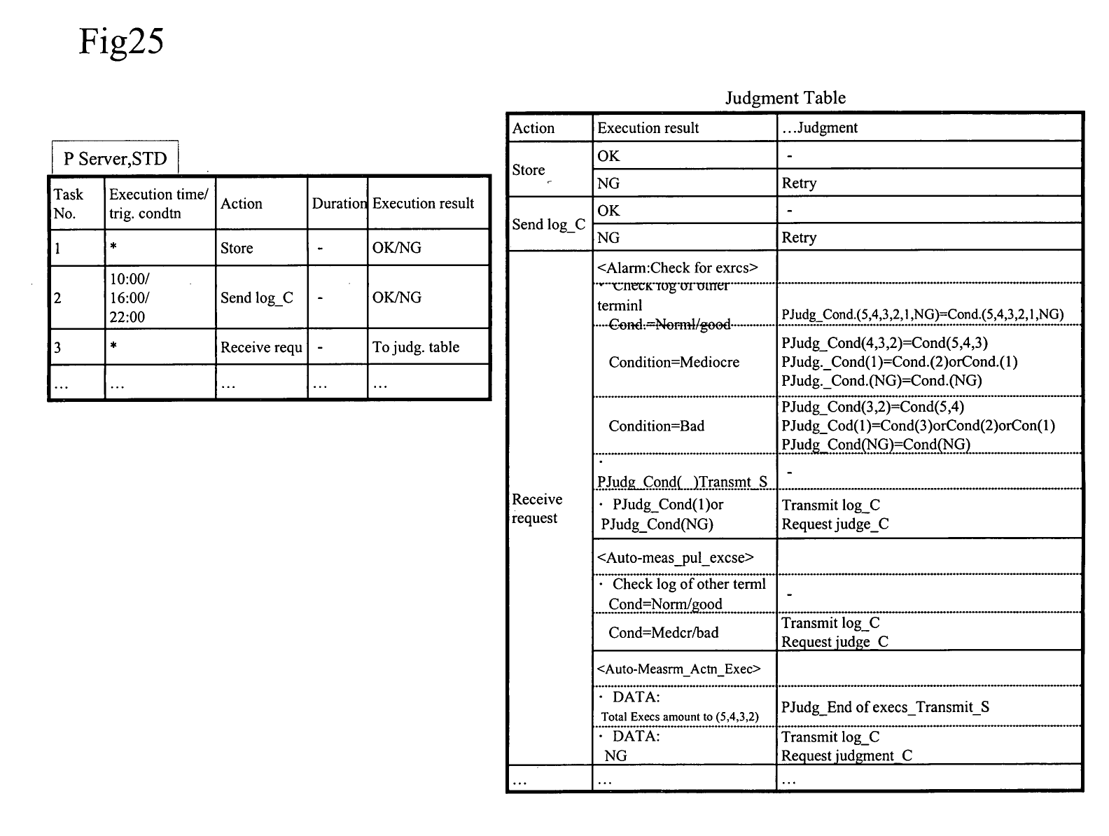

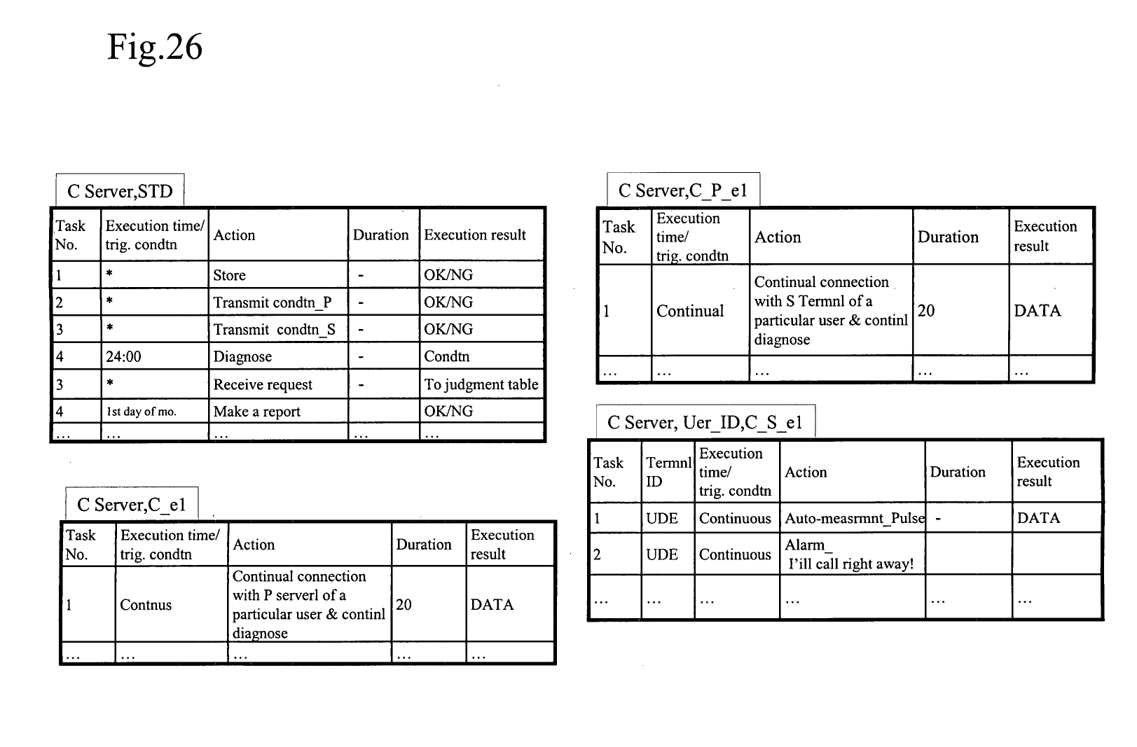

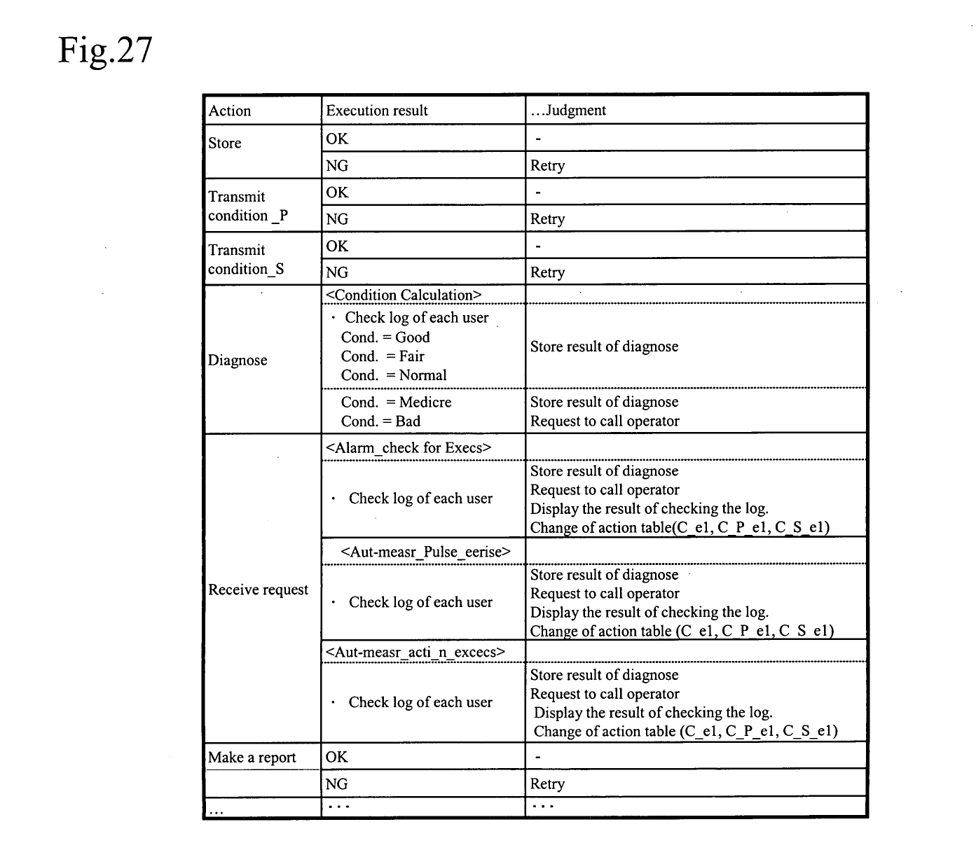

Next the action table information and the judgment table information are explained which are stored in a living-body information terminal, P Server 4, C Server 2. FIG. 24 is a figure of the action table information and the judgment table information which are stored at the living-body information terminal, FIG. 25 is a figure showing the action table information and the judgment table information which are stored at the P server 4, and FIG. 26 is a figure showing the action table information stored at the C Server 2, FIG. 27 is the judgment table information stored at C Server 2. The action table information and the judgment table information shown in FIG. 24 are stored at each living-body information terminal.

When judgment has to be made, the judgment table information of its own is used, And when judgment is difficult, or when it needs to judge from higher point of view, for example, when the processing time of judgment takes too long, or it is necessary to judges with reference to two or more databases, etc. it requires judgment of a higher rank, server then, it transmits request to the higher rank server for judgment with a transmission log. For example, each living-body information terminal requests to P Server 4, or C Server 2 for judgment, while P Server 4 requests to C Server 2 for judgment.

The process of health-care system is explained at the stage where table information depicted in from FIG. 24 to FIG. 27 are stored. Here in the standard action table information of the living-body information terminal (FIG. 24), the terminal ID “UDE” corresponds to the wrist watch type terminal 101, and the terminal ID “MM” corresponds to the portable type terminal 102. At 10 o'clock, following the task 1, the wrist watch type terminal 101 informs the alarm for the start of exercise, and displays a message to urge the user to input the user's condition. One the user selects one of five grade, the wrist watch type terminal 101, based on the judgment table information, sends to the P Server 4, a log and a request for judgment.

P Server 4 receives the log sent from the wrist watch type terminal 101 and sores the log (corresponding to the task 1 of the standard action table information of P server 4 in FIG. 25). Responding to the transmitted request for judgment, the P server 4 detects one of “Good”, “Fair”, “Normal”, “Mediocre”, and “Bad”, from the condition level received as a log, and the judgment table information in FIG. 25. and sends the detection result to the wrist watch type terminal 101 and portable type terminal 102. When the detection result is “bad”, P Server 4 sends the log which says the condition data is bad, to C Server 2, and demands the judgment. As the result, in C Server 2, the action is performed by prompting the operator to make phone call to the user (following the judgment table information in FIG. 27).

The wrist watch type terminal 101 and the portable type terminal 102, receives the diagnostic result of the condition level transmitted from P Server 4. In the case user's condition level is “bad”, the wrist watch type terminal informs that the user is not to do exercise, and that the user will receive message from the operator by telephone or by others (corresponding to Task 6, FIG. 24). On the other hand, when the condition level is “Good”, “Fair”, “Normal”, “Mediocre”, the wrist watch type terminal 101 informs the alarm of start of exercise (corresponding to Task 3, FIG. 24). At the same time, the portable terminal 102 also informs that the exercise is to be started (task 101, FIG. 24).

The portable type terminal 102, measuring speed, and angular velocity, and computes the amount of movements based on the detected measurement and when the total amount of movement reaches predetermined value, sends the total amount of movements to the P sever 4. Following the judgment table information of FIG. 24, the P Server 4, after judging that the completion of the exercise by the judgment table information of FIG. 25, informs the end of the exercise to the wrist watch type terminal 101, and the portable type terminal 102. On receiving this notice, the wrist watch type terminal 101, and the portable type terminal 102, inform the user the end of the exercise (corresponding to Task 5, and task 103, FIG. 24).

The wrist watch type terminal 101 automatically measures pulse during exercise (task 4 of FIG. 24), and accumulates measurement data. When abnormalities are detected by measurement data, the wrist watch type terminal 101 sends a log indicating the detection of abnormality, and request for judgment to the P Server 4. The P Server 4, based on the judgment table information of FIG. 25, sends this log to the C Server 2, and requests judgment to the C Server 2. The C Server 2, based on the judgment table information of, FIG. 27, stores this log, and at the same time, informs to operator to call the user. Then the action table is changed to the exception action table of FIG. 26.

In the case that in the action table information, actions for the sensor terminal 103, and the desk top type terminal 104 are defined, various measurements are performed following to the action, the log of the result of the measurement is made and stored. For example, the P Server 4, when prompts the wrist watch type terminal 101, to inform the user to take medicine, makes the sensor terminal 103 to detect whether the user is at sleep. And if the user is at sleep, the server delays the execution time of the action by changing the action table information of the wrist type terminal 101, so that the user can take medicine after the hour of rising. The change of the action table information is sent from the P Server 4 to the C server 2 and stored as a log. In this way, it is possible to use two or more living-body information terminals in a coordinated way. Based on these results, or detection results, it is possible to perform various cares.

Additionally P server 4 may have the function to transmit and receive data with the electrical equipments at home having the communication function. For example, P server 4 knows that there is a visitor by detecting a visitor by an interphone, P server 4 informs the wrist watch type terminal 101 that there is a visitor. P server 4 may unlock the door key when the direction of the unlock is inputted to the wrist watch type terminal 101. Moreover, the wrist-watch type terminal 101 may have a function of the remote control of the television. It may be provided to an physically handicapped user as a user interface device. P Server 4, by over viewing the detection results of each living-body information terminal may control the setting of temperature of air blow of an air-conditioner, and etc.

In this embodiment of the present invention, the following processings are possible.

(1) The wrist watch type terminal 101 notifies to a user the time of blood sugar measurement performed by desk top type terminal 104.

(2) The P Server 4, from the data from the sensor terminal 103, creates the compensation coefficient for rectifying measurement data of pulse for the wrist-watch type terminal 101 measurement, and transmits the compensation coefficient to the wrist-watch type terminal 101.

(3) After directing the user to perform exercise, the analysis at P Server 4 is made on the measured pulse data collected by wrist watch type terminal 101, and the amount of movement data collected from the portable type terminal 102. And P server 4 decides from the result of the analysis, the time of the next medication, the amount of medicine, and next measuring time to notify to the user at later time.

(4) The P Server 4, and C Server 2 update the action table information so that either wrist watch type terminal 101 or the sensor terminal 103 can measure the pulse.

In the second embodiment of the present invention, since the frequency of communication between the P Server 4 and C Server 2 can be reduced, it is possible to use a dial-up system at the communication line 3. Moreover, even in cases such as communication failures in the system or users being outside of the communication boundary, the system can operate normally at the abnormality of a user since the action table information is stored in the living body information terminal and P Server 4. And communication between the P Server 4 and each living-body information terminal can be made denser, fine control can be performed according to changes of a user's physical condition.

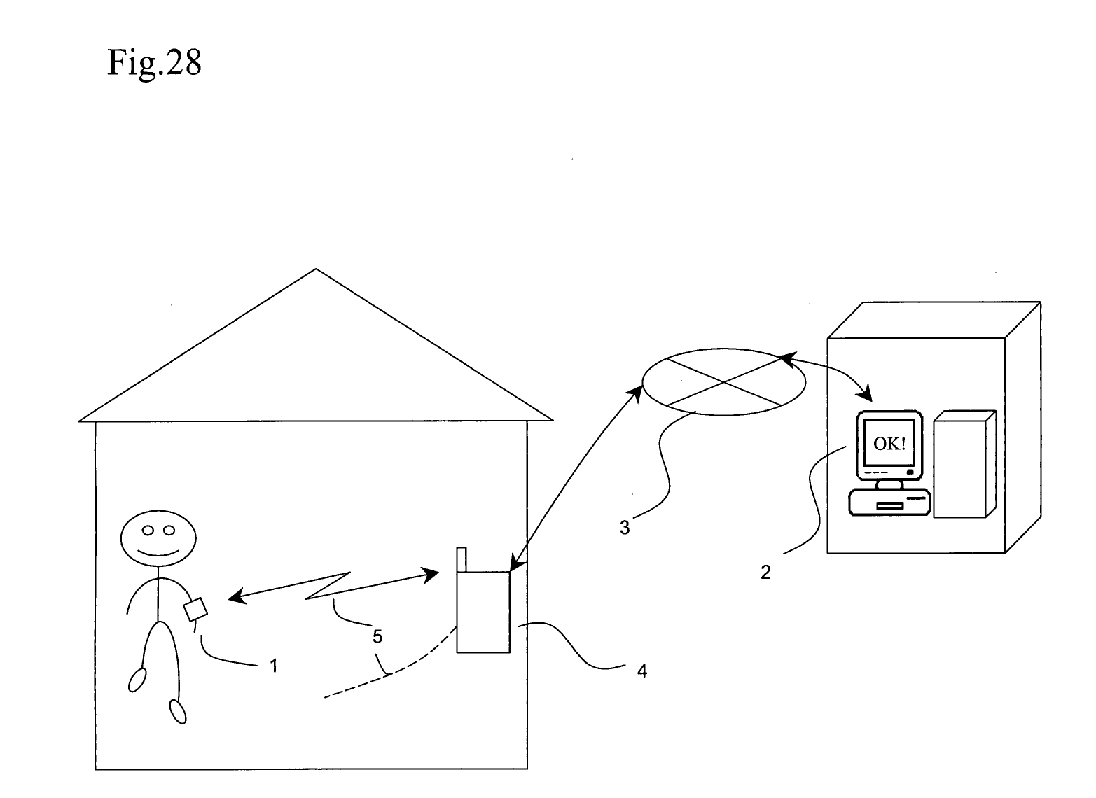

In the above-mentioned second embodiment of present invention, the case where two or more living-body information terminals were used was explained. As depicted in FIG. 28, only one living-body information terminal (here wrist watch type terminal 101) may be used.

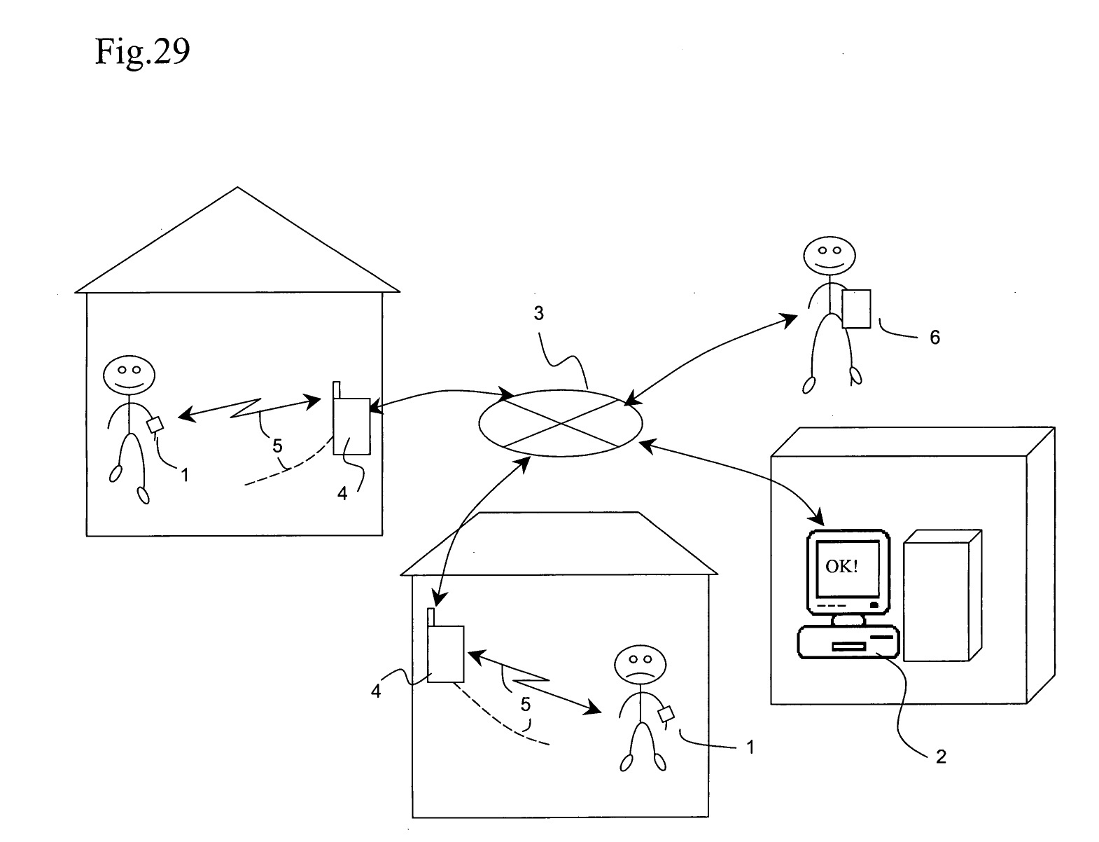

Next, the third embodiment of present invention is explained. FIG. 29 is an outline block diagram to show the composition of the health-care system in the third embodiment. In this figure, the information terminal 6 is held by a home helper, a nurse, the doctor, etc., who visit the home of a user, communicates bi-directionally with each living-body information terminal via the C Server 2. According to this embodiment of the invention, it is possible to perform following processings.

(1) A user sends, using a living-body information terminal, to other users in a predetermined group though the communication line 3 the health condition information on the day (character information, measurement data of pulse or temperature), receives other users' health condition information, shares health information each other, and recognizes each others' health conditions.

(2) Each living-body information terminal, following the action of the action table information, sends the message “How are you?” to other living-body information terminals in a predetermined group. The C Server 2 gathers the answers, sends the result to the terminal 6. A home helper, with the information terminal 6, can acknowledge the condition of the user, and send messages to a user if necessary, asking for the condition.

(3) In the C Server 2, if it is detected that the user's condition is not so good, the server can send a message to the information terminal own by a family member, to encourage to make phone call to the user.

In the third embodiment of the present invention, it is possible to offer the service using the result derived from the comparison of a user's condition data with the ones of the users of other areas. Moreover, it is possible to grasp about a user's condition in the distant place, in this embodiment, a user can be contacted only at the time of an abnormal condition, the family who lives away from the user is able to live in comfort. Moreover, according to this embodiment, since it may be possible to share health condition information of users with similar health condition, users can communicate each other, and the anxiety against diseases and health condition can be lowered.

Although changes of the action table information explained in embodiments are made according to the execution result of action following the contents defined in the judgment table information, it may be made to change, for every predetermined term, by the person (a doctor or operator) to whom access was permitted.

In addition, in the embodiment mentioned above, only ID of action table information may be transmitted, and the action table information may be changed to that corresponding to the ID. Moreover, the second schedule information may be made changed in such a way that a part of the first schedule information is changed.

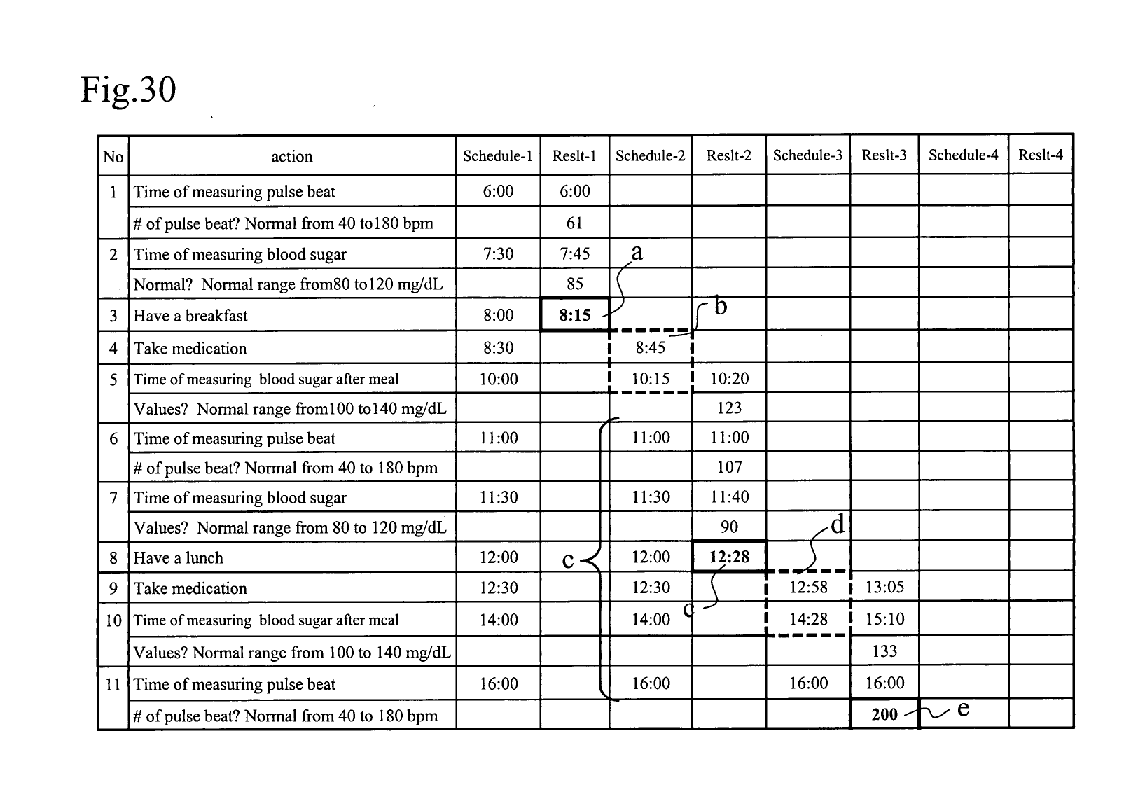

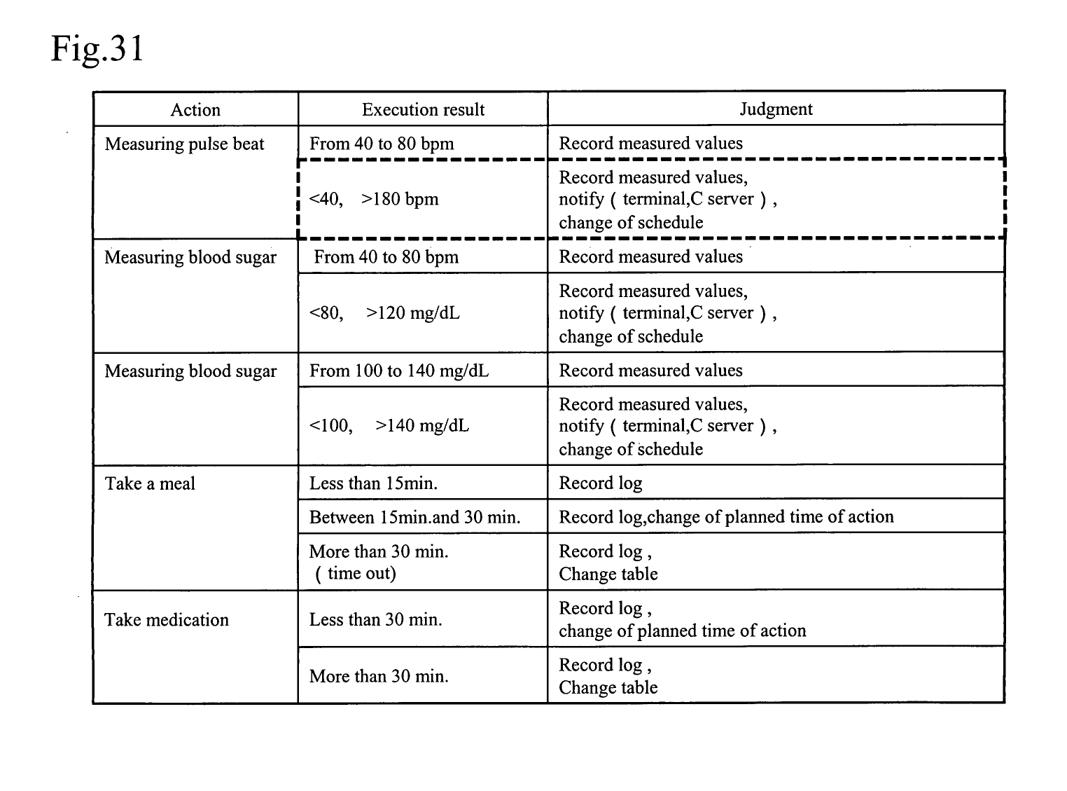

Next, the fourth embodiment of the invention is explained. In this embodiment, the case is explained where the action table information is changed based on the detected result of information about a user's condition at the S terminal 1. Here, while the action table information shown in FIG. 30 is stored in the memory part 14 of the S terminal 1, the judgment table information shown in FIG. 31 is also stored in it.

Action table information contains a time and a related action, which should be made at the time. Moreover, in this embodiment, the action table information contains a standard action table (for example, the column of “schedule-1” in FIG. 30) as well as the action table information after changes (for example, the columns of “schedule-2”, the “schedule-3”, and “schedule-” in FIG. 30). After this change, data is written in the action table, according to the direction from the Schedule Execution Management part 15.

The judgment table is the information contains an execution result of the action taken according to the action table information, and the next process corresponding to the execution result. In the judgment table, actions corresponding to lapsed times, as well as measurement result are specified.

In this embodiment, the Schedule Execution Management Part 15 changes a part of the action table information if needed, referencing to the judgment table information, based on the execution result of action performed according to the action table information. This change is also made to a part of the action table regarding to other actions relevant to this action which produces an execution result causing changes necessary.

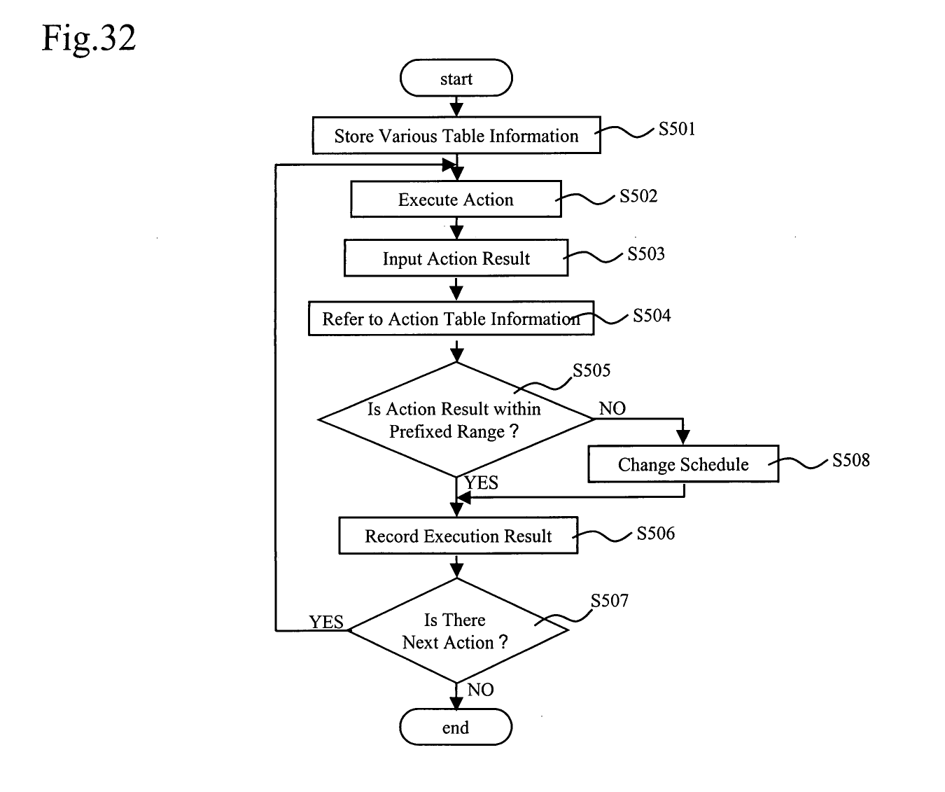

Next, the case is explained where these action table information and judgment table information are applied, using the flow chart of FIG. 32. First, the action table information shown in FIG. 30 and the action table information shown in FIG. 31 are received from the P server 4 or the C server 2 via the P server 4, and are stored in the memory part 14 of S Terminal 1 here (Step S501). Next, the schedule execution Management part 15 reads the action table information, and performs action which responds to the present time (Step S502). And the execution result of the action is inputted (Step S503), referring to the judgment table information (Step S504), the schedule execution management part 15 detects (Step S505), whether the execution result of the action is within the regulation values shown in a judgment table. When the execution result is within the regulation values, the execution result is stored as a log. When it is outside of the range of regulation values, the execution result is recorded (Step S506) after action table information is changed (Step S508). And it detects whether the following action is in the action table information (Step S507) and if there is the following action, it will shift to Step S502 and will perform according to the execution time of action, and if there is no action specified, it will end.

Next, change of the action table information by the schedule execution Management part 15 is explained further. The schedule execution Management part 15 performs according to the schedule-1 in the action table information in FIG. 30 at present time, performing pulse measurement, blood sugar measurement, and others one by one, and stores an execution result to the column of “result-1” in the table. And in the action of “Have a breakfast”, when the time of taking breakfast is inputted as “8:15”, the regulation value corresponding to the item of the judgment table of FIG. 31 “take a meal” is referred to. Here, the difference of the regulation value in the standard action table and the execution result is computed, the management part refers to the regulation value corresponding to the difference. Here, since the difference corresponds to “more than 15 minute and less than 30 minute”, while the execution result of action is stored (FIG. 30 mark a), action schedule time is changed according to the difference. Here, since the amount of difference was 15 minute, actions of “Take medicine” and “Time of measuring blood sugar after meal” triggered from the action of “take a meal”, are set at the times which correspond to the difference, and the times are stored into the column of “schedule-2” (FIG. 30 mark b) of the action table as information of action table after the change. Actions other than the item changed here, actions set in the standard action table information are used (mark c). Henceforth, action is performed according to the information in “schedule-2”, referring to the judgment table information, and the action is performed according to the corresponding regulated values range. And when change arises to action table information (FIG. 30 mark c), the time corresponding to the difference between scheduled time of the action at the time and the time of the execution result is set at the column of “a schedule-3”, as the action table information after change (mark d).

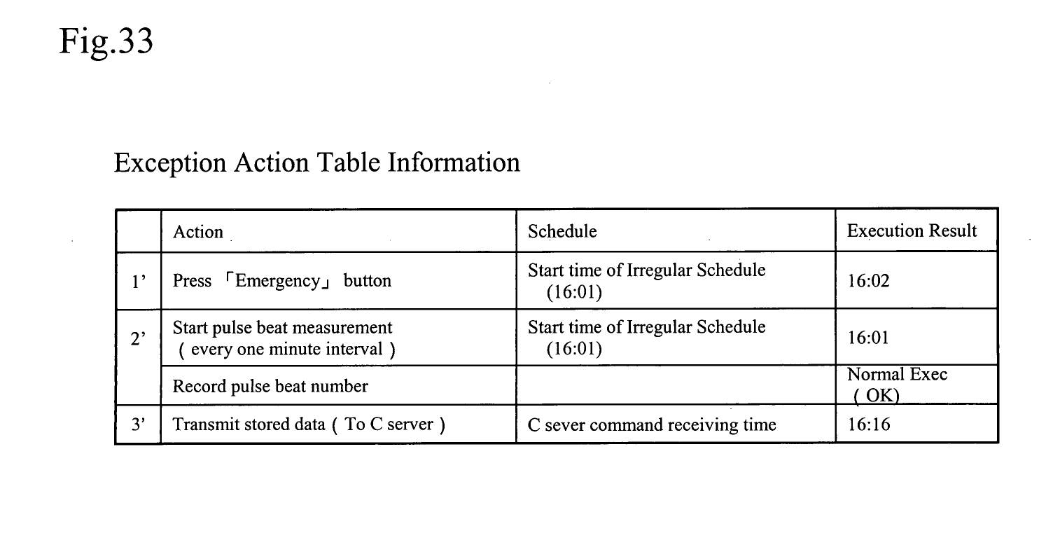

On the one hand, when the execution result of action is not time but values such as blood sugar value, and the value is outside of the regulation value range (mark e), since this case corresponds to the item of more than 180 bmp (>180 bpm) in the item (measure pulse) shown in FIG. 31, the measured value is reported to a terminal (P server 4) and the C server 2. And action table information is changed into the exception action table information shown in FIG. 33. Exception action table information will be started at 16:01 in response to the measurement result at 16:00 shown in FIG. 30 mark e. And an urgent button becomes active with the notice of “pushing an urgent button”, and pulse measurement is started. And the measurement result is recorded and according to the directions from the C server 2 is transmitted to the C server 2. If an urgent button is pushed by the user, the operator of the C server 2 will be notified. Thereby, an operator contacts to the user using a telephone etc., and he can correspond like measurement continuously for 15 minutes and keep on observing and etc. And after at 15 minutes after the start of measurement, at 16:16, the C server 2 sends out a data transmission command, thereby, a measurement result is transmitted from the S terminal 1 to the C server 2. The S terminal 1 reads the action table information on “a schedule-3” after measurement result transmission. After checking this measurement result, the operator consults with a specialist etc. if needed, and contacts a user again, and reports the measurement result and the result of the consultation.

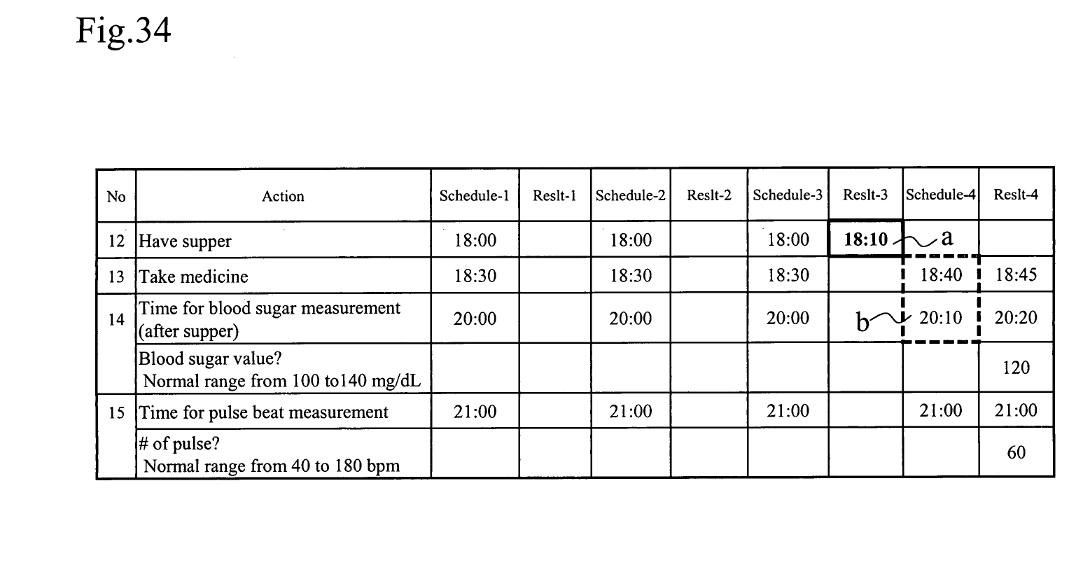

And as henceforth shown in FIG. 34, actions are performed according to the action table information in “a schedule-3” and if the execution result is outside of the regulation value range (FIG. 34 mark a), the action table information after the change is set as “a schedule-4” (FIG. 34 mark b).

In addition, in this embodiment, as in the previous embodiment, laps times of the use's input regarding the action may be managed. Moreover, besides time and living body information data, the fact that actions are taken according to action table information, (as have measured or alarmed) may be managed.

Since the schedule execution management part 15 can change a part of action table information, if necessary, referring to the Judgment Table Information responding to the execution result of action, without being transmitted the changed action table information from C Server 2 or P Server 4, in the S terminal 1, the action table information can be changed.

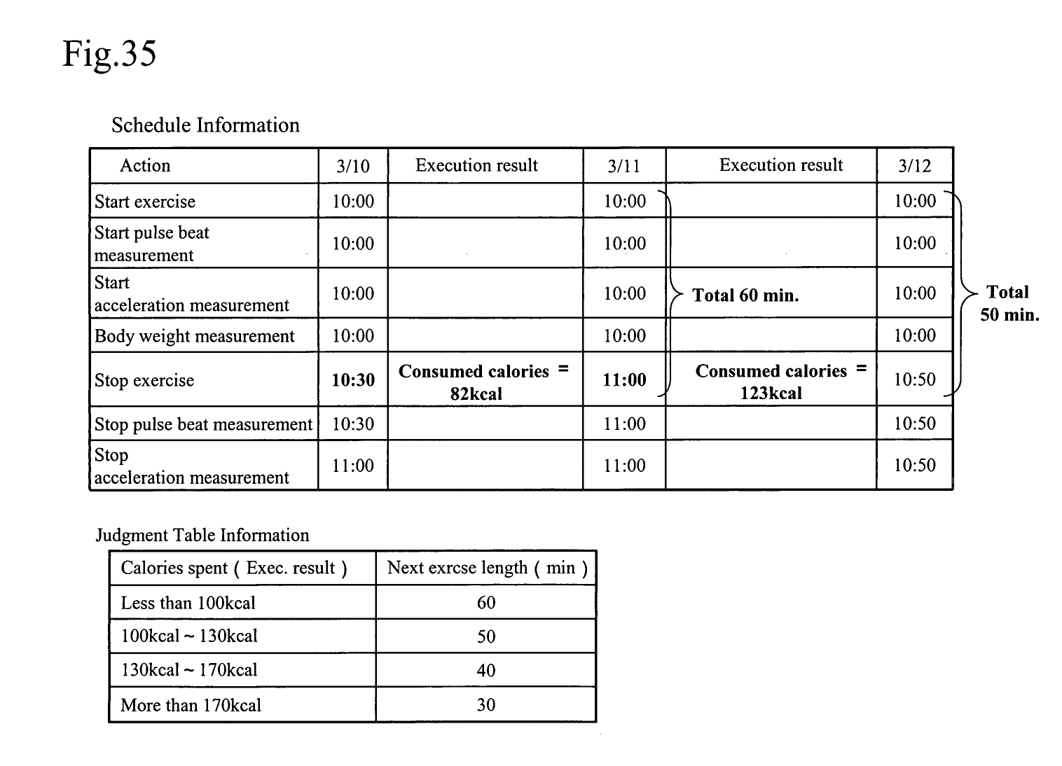

Next, other embodiment is explained in which the action table information is generated, using FIG. 35. FIG. 35 shows the action table information showing the schedule for having a user exercise, and the judgment table information defining the next movement time which embraced the actual result of the consumption of calories by this movement. These action table information and judgment table information are stored in the memory part 14. And when this action table information is performed, the schedule execution Management part 15 computes the actual value of consumed calories from a start to an end of the movement and reads the next movement time, which corresponds to the computed value from judgment table information, and will set up a next movement time. Here, since the actual result value of 3/10 (March 10) of consumption calories is 82 kcal, based on judgment table information, the next movement time of 3/11 (March 11) is set as 60 minutes. And when the actual result value of 3/11 of consumption calories was 123 kcal, with reference to judgment table information, movement time of 3/12 (March 12) is set as 50 minutes. Thus, in this way, it is possible to change the action table information about next movement according to the amount of movements.

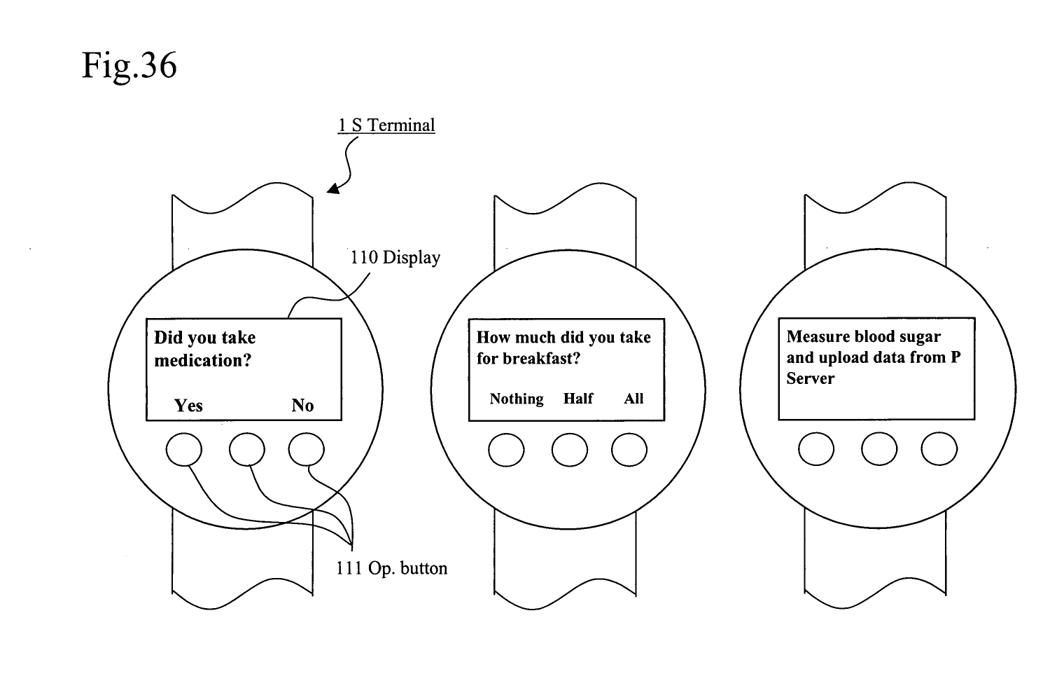

Next, the S terminal 1 in the fourth embodiment is explained using FIG. 36. FIG. 36 is an outlook composition figure showing the appearance of the S terminal 1. In this figure, the S terminal 1 has a display part 110 (noted as “Display”), which displays the contents of action and choices for a reply, and an operation button 111 (noted as “Op. button”) for inputting a reply corresponding the choice. Here, information which should be displayed according to action to be performed is added to action table information, and is transmitted from the C server 2 or the P server 4, and is stored in the memory part 14. And while the information for urging to act like “Did you take medicine?” according to performed action is displayed, the choice which responded to the action, “YES” and “NO” are displayed near one of operation buttons 111. Here, the choice and the near operation button 111 are corresponding and if the operation button near the “YES” is clicked, the information of “YES” will be inputted into the S terminal 1.

Moreover, with software, which only activates the operation button corresponding to choice, the central operation button 111 does not become active, since there is no corresponding choice in FIG. 36 (a). For an action shown in FIG. 36 (b), the choices for the action of “how much breakfast was eaten” are “non”, “a half”, and “whole” are displayed, thus all of three operation buttons 111 become active. Moreover, as shown in FIG. 36 (c), it is also possible to display only the guide information like “please measure blood sugar value and upload data from P server”. In addition, in this case, it is possible to manage the above mentioned timeout to operate the operation buttons 111.

In this embodiment, the action table information in a certain S terminal 1 may be displayed at other S terminals 1, or an execution result may be displayed. In this case, in response to the demand from another S terminal 1, an S terminal 1 accepting the demand, may transmit information corresponding to the demand, or another S terminal 1 may access the memory part 14 of this S terminal 1.

In this case, since each operation button 111 is controlled to make it active or not by displaying selections corresponding to operational buttons 111 of the S Terminal 1, it is possible to utilize effectively the operation button 111, by assigning a command to the operation button 111 dynamically.

Next, the fifth embodiment is explained. Here, the case is explained where action table information is not changed according to the detection result from a living-body information terminal, but is changed according to the directions from the exterior. (Let henceforth the action table information be the third action table information which is changed according to the directions from the exterior)

The C server 2 obtains temperature information periodically. For obtention of weather information, several ways can be considered; using a public line from a weather information distribution company, the communication part 21 receives information, the temperature of the environment in which a user is, is obtained from the P Server 4, by installing a temperature sensor in the P Server 4, by putting temperature sensors by or in user's house, and that temperature information is received from the P Server. Of course, a temperature sensor can be installed in the C server itself, and the temperature information may be referred to.

And when temperature of obtained weather information is lower than the temperature decided beforehand, the C server 2 changes the action table information in a living-body information terminal into the third action table information with the content of increased number of time of measurement of pulse. Here as the changing methods of action table information, first method is whenever change is needed, the C Sever 2 transmits the third action table information for living-body information terminals to the S terminal 1, or in the second method, all action table information is beforehand transmitted and stored in the S terminal 1, and when change is needed, the C server 2 transmits ID which shows either of the third action table information stored in the S Terminal 1, and directs change of the action table information.

Especially, it is possible for the C Server 2 when the S Terminal 1 is turned on, on the next day, to transmit the changed action table information as the first action table information (standard action table information), when the weather of the next day can be told as bad as today from weather information. Moreover, a user can check the difference between the contents of the schedule (action table information) of today, and the schedule of yesterday by notifying the user of the action table information of the day by the P server 4 or the S terminal 1 at the time of starting S terminal 1.

Although only the case where natural phenomena, such as the weather causing the change of the action table information is explained, but the action table information can be changed for each case according to situations, such as a calamity, as other natural phenomena. In order to prevent consumption of a battery, at the time of a calamity, a user can be navigated to the refuge place, and measurement frequency may be lowered here, until the user could reach a refuge place.

Next, an example is explained where the fifth embodiment is applied. Here, when a user arrives at an airport, an action table information for airplanes will be transmitted to a user's S terminal 1 from the P server 4 installed at the airport.

Here the action table information for airplanes is, for example, in consideration of deep-vein thrombosis (economy-class syndrome), is an action table information in which measurement frequency may be raised, and also includes an extraordinary connection setup information of the super-short-range wireless communications or of the wired communication via a communication jack at the seat. Moreover, also inside of a plane there shall be another P server 4 that collects the information from the S terminal 1.

When abnormalities (as in a case where living-body information data from measurement exceeds the threshold value decided beforehand) arise to living-body information data of a user in a plane, a passenger attendant is notified of abnormalities having arisen to a user by the communication from the S terminal 1. Thereby, a emergency measure can be given to the user. Moreover, when a judgment can be made that a user has fallen asleep, by S terminal 1 or the P server 4 on board of a plane, from stable values of living body information data (almost static state in measured values), the P server 4 on board the plane may carry out fade-out of the volume of the head phone the user is using, and may lower reclining of the chair, by transmitting commands to the sound equipment and the chair control device in an airplane. Thereby, a user can have good sleep.

After landing, when the end signal of the action table information for the inside of an airplane is transmitted to the S terminal 1 from the P server 4 of an airport, the S terminal 1 returns to the first action table information, and notifies the user of having returned to the first action table information, by using the display screen of the S terminal 1.

In addition, although an example in which action table information is changed according to places such as airport and airplane, it is also possible to change action table information individually according to places like outdoor, specific indoor place (a bathroom, a bedroom, etc.), and specific institution (a hospital, a department store, a school, in a company, community, etc.). Here, examples of the third action table information, can be given as follows;

(1) in a bathroom, blood pressure, pulse, and room temperature can be measured more frequently.

(2) in a bedroom, the living body information data can be measured more frequently by a stationary sensor.

(3) in a hospital, communications are stopped.

(4) while in a department store, more frequent measurement of the pulse.

Next, another example of the fifth embodiment is explained. When a doctor needed to visit user's home to diagnose, and needs to perform various measurements there, the doctor accesses to a P server 4, through his/her own portable terminal, and the doctor is able to create the action table information for extraordinary measurement (in order to measure transitional change of pulse rate under exercise load, information directs continuous measurement of amount of movement and pulse rate, and continuous data transmission to P Server 4, and to the doctor's portable terminal), can register it with the P server 4.

When the action table information for extraordinary measurement is registered into the P server 4, the action table information for extraordinary measurement will be transmitted to a user's S terminal 1 and a doctor's portable terminal, and if it is registered into a user's S terminal 1 and a doctor's portable terminal, they will be in the state in which extraordinary measurement is possible. A doctor directs various operations (movements) to a user, can observe changes of the amount of movement and pulse rate on that occasion, can diagnose the user, and the better diagnosis can be done. Each terminal and equipment return to the first action table information after the diagnosis ends.

As the result of the diagnosis, if a change is needed for the kind of medicine to take, and time to take it, a doctor can access the P server 4 through his/her own portable terminal, and can change the medicinal kind and the medicinal recipe time in the first action table information of a user. When registration of change is completed in the P server 4, the P server 4 notifies with a sound, the changed part (the recipe time change) of the changed action table information, and transmits the changed action table information to the user's S terminal 1 and C server 2.

Although the example in which action table information is changed by a doctor (or specialists, such as a care giver and a nurse) is given, in other examples, it is also possible to change action table information individually according to the human factor by the user him/herself, a relative, a friend, an operator, a system manager, and etc. Here, a user him/herself, a relative, and a friend may be able to increase the number of kinds of measuring instrument, and the number of times of measurement. Moreover, a terminal to receive notice and alarm can be a terminal possessed by a medical related people, a care giver, a seeing eye dog, and a superior official in addition to S terminal 1 and C server 2.

Next, explained is other embodiment of the C server 2 as sixth embodiment. The feature in this embodiment is that the C Server 2 has an analysis function to analyze living-body information data stored in the C server 2 transmitted from the S terminal 1 over a long term. In this embodiment, the action Management Part 25 of the C server 2 has the analysis function to analyze in a predetermined term based on the history of living-body information data which was transmitted from the S terminal 1 and stored in the memory part 24. This analysis function can be roughly divided into two elements as shown below.

(1) Even if the value of living-body information data is not over the maximum value decided beforehand, it detects whether there are any signs of abnormalities and it changes the kind and its contents of action table information when there are signs of abnormalities.

(2) When regularity is detected in living-body information data with a fixed cycle, it changes the kind and its contents of action table information according to the regularity.

Explain hereafter are each example of above-mentioned cases (1) and (2).

An Example of (1)

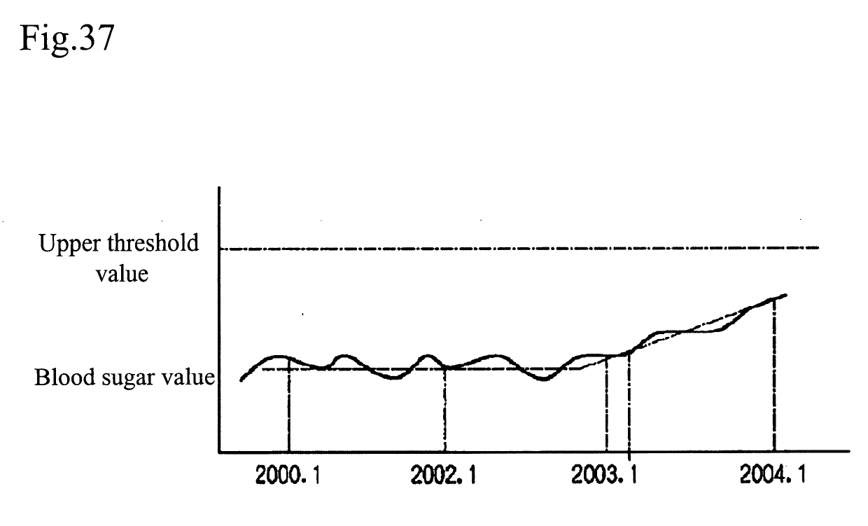

When a user measures blood sugar values, weather the blood sugar value transition is in an uptrend, stabilized (or almost constant), or in downward tendency is detected in every three months. In this detection, for example, when the history of the living body information data transmitted from S Terminal 1, is like the graph as shown in FIG. 37 the action Management part 25 computes inclination of the history for every fixed term, and if the calculation result is bigger than the value decided beforehand, it detects as a thing with the signs of abnormalities. And in the case where it detects signs of abnormalities, the kind of action table information is changed for a person with a high blood sugar value. Here, the information for carrying out change of the kind of medication, change of the amount of medication, change of medication time, the increase in the amount of target movements of movement performed every day, etc. as action table information is included. Moreover, when signs of abnormalities are detected, while the doctor in charge is notified here, the message such as “Blood sugar value is going up little by little. Please be careful”, are displayed on the S terminal 1, and it warns him/herself. In this way, by referring to living-body information data in a long term, before the condition gets worse, a doctor and a user are able to cope with the situation.

An Example of (2)

Here, when a user measures the pulse rate, by observation of the pulse rate measured every day for a fixed term (for several years), the action Management part 25 analyzes the feature of the transition of pulse rate, and detects tendency. For example, when it detects the tendency that pulse rate is high between June and August, or when the temperature is higher than 27 degree C., afterward, it changes the kind of action table information, for the month, and for the temperature, if the communication parts 21 receives that the time has come to the month or the temperature becomes to the temperature (between June and August, or the temperature of 27 degrees) corresponding to the tendency. This action table information includes information for increase of the number of measurement of pulse rate and for decreasing the amount of target movements performed every day for example. Furthermore, in this case, the user is warned by messages as “Be careful since it is June”, or “Be careful since temperature is high” displayed on the S terminal 1. Thereby, the intricate health management specialized for an individual user is attained.

In this embodiment, the time to detect the tendency of living body information data may be at rising time (at first communication time of S Terminal 1), at weekend, at a weekday, at certain time while out of bed, at a time in the morning, or in the afternoon, at a season etc. for example.

In the fifth and sixth embodiments, when the schedule changed according to a direction from exterior, or the analytical result of the loving body information data analyzed over long period, is executed, the schedule may be further modified to another schedule according to the execution result of the modified schedule. Thereby, the intricate physical condition management corresponding to changes of the environment of a user's circumference, or the tendency of the physical condition can be carried out.

Next, explained is the seventh embodiment. In this embodiment, the program (software) for realizing the function of the P server 4 may be downloaded to the cellular phone, which has a short-range wireless communications function, and it may be made to perform the program which is downloaded, by a cellular phone. The information containing the measurement result which is measured at the S terminal 1, regardless of a place, can be transmitted to the cellular phone used as the P server 4.

Moreover, when there is information which it is urgent and is notified from the C server 2, it is enabled to correspond by communicating with a cellular phone. Moreover, information (for example, the contents of a meal, position information, etc.) effective in a health-care system can be inputted by using effectively the function which a cellular phone has.

Moreover, the program (software) for realizing the function of the P server 4 is downloaded not for a cellular phone but for the indoor installation type product (for example, television, a refrigerator, the product installed in the toilet, the product installed in the bath) which has a short-range wireless communications function, and it may be made to perform it. Thereby, regardless of an indoor place, since the information containing the measurement result measured at the S terminal 1 can be transmitted to the C server 2, when the S terminal 1 needs to inform a user of information, the contents of a notice can be notified using an indoor installation type product. It is possible to display by title display of television or to carry out a audio output by speaker of various products etc. as the method of a notice. Thus, when state of emergency arises to a user by notifying using various products, it can notify that state of emergency has occurred for the indoor installation type product of the place in which an inmate is present.

Moreover, the program (software) for realizing the function of the C server 2 may be downloaded to other apparatus which have a communication function. For example, it also becomes possible to realize further health-care business by downloading and performing to a general-purpose computer by providing with software the contractors (for example, a public organization and medical organization, a health-care provider, a medical goods handling store, etc.) who think that he wants to perform health-care business mentioned above.

For example, it can be used for the member of rescue team in the calamity generating spot. Here, software can be downloaded to the computer brought to the calamity spot, and member's healthy condition and the priority of rescue etc. can be analyzed by making a calamity rescue member carry a living-body information terminal.

Functions of the living-body information terminal communication part 11, the time recorder 12, measurement part 13, schedule execution management part 15, report part 16, operation part 17, control part 19 in FIG. 2, functions of the communications part 21, the time record part 22, data-processing part 23, action management part 25, information part 26, operation part 27, and control part 29 in FIG. 4, functions of the communications part 41, time record part 42, data-processing part 43, schedule execution management part 45, information part 46, operation part 47, and control part 49 in FIG. 21 are programmed and stored in a computer readable record medium, and by inputting this program to a computer system, and running the program in the computer system, it is made possible to manage the schedule. The computer system herewith includes programs as well as the operating system (OS), hard wares including peripherals.

And also, if it is the case where the system “computer system” uses WWW system, homepage offer environment (or display environment) shall also be included.

The computer readable record medium mean a memory includes portable medium such as a flexible disk, a magneto-optical disc, ROM, and CD-ROM and also includes a built-in hard disk in a computer. Also the computer readable record medium includes mean to hold dynamical program for a short time like the communication line such as a network of the internet and etc., or communication lines such as telephone circuit, and includes mean to hold the program for a fixed time like the volatile memory in the computer system. The said program may be to realize a part of the said functions, or may be that the said functions are realized combined with another program already stored in a computer.

Although the embodiments of present invention has been explained in full detail with reference to drawings, concrete composition is not restricted to these embodiments, and a range of design which does not deviate from the gist of this invention etc. is included in present invention.

### INDUSTRIAL APPLICABILITY

As explained above, with according to the present invention, since the schedule information can be changed according to the execution result of the action following the first schedule information, an appropriate schedule information according to health condition, action or disease condition of user can be set at the living-body information terminal

Since the schedule information can be changed according to directions from exterior, schedule information at the living-body information terminal can be set corresponding to changes of environment of a user. And even schedule information at the living-body information terminal is set according to external environment, schedule information can be further changed according to the detection result of a detection means.

The schedule execution result following the schedule information includes whether action following the schedule information was taken, or whether action was taken as well as its execution time, or measuring result of health condition, or measuring result of health condition as well as its measuring time, or inputted information inputted from an inputting means, or inputted information inputted from an inputting means and its inputting time, or whether detection is made within the predetermined continuation time of the detection of whether action is made, and since such detail information is included, based on the information, more user dedicated and more detailed schedule information can be made and executed.

Since it is made possible to change schedule information based on identification information by sending a necessary schedule information of a living-body information terminal before hand, information volume of communication between a healthcare device and a living-body information terminal can be decreased.

And since the second schedule information and the third schedule information dealt in the health-care device of the present invention is information changed a part of said first schedule information, the volume of the second schedule information or the third schedule information is decreased, thus information volume of communication between the health-care device and living-body information terminals can be decreased. And, the volume of storage of schedule information in the memory part of a living-body information terminal can be decreased.

It is possible to provide user dedicated preparations and measures at the living-body information terminal by judgment based on judgment table information. Since only when specialist judgment is required, judgment request is made to health-care device, thus unnecessary communication contacts to doctors by connecting communication line at a distance for every small abnormality can be avoided. And power consumption of the living-body information terminal regarding communication can be reduced. Furthermore even at the absence of the doctor or the living-body information terminal out of communication range, an appropriate service can be provided by the living-body information terminal by itself.

A user can see at his most convenient living-body information terminal, schedule information of other terminal and execution result of schedule.

Since schedule information for an individual user can be appropriately made or changed, for the user with chronic diseases, such as diabetes which requires long term health management, or cardiopathy which requires regular surveillance, it can provide individual care, and make sure whether schedule information is followed. Moreover, without close contact with a patient, and a continuous and accurate measurement inspection can be conducted from distance to the patient with infection, such as SARS (serious acute respiratory-organs syndrome).

Moreover, since schedule information and judgment table information are information which can be treated as a table, and the display, create, and change of the contents can be made very simple, creation and change of a schedule are very easy also by the man who does not know program language at all as compared with the case where the contents of schedule information or judgment table information are programs which can be executed by a computer. Moreover, since it can check as a table, unlike seeing the source code of a program, it is very easy to grasp the contents.

1-54. (canceled)

55. A health-care device that communicates with a living-body information terminal adapted to detect health condition information concerning a health condition of a user or to issue action directions to said user to take action regarding a medical treatment, comprising:
schedule storage means for storing a first schedule information for said living-body information terminal to detect a health condition information of said user or to issue an action direction to said user to take an action, and for storing a second schedule information to be executed by said living-body information terminal in response to an execution result of said living-body information terminal following said first schedule information;
communication means for reading said first schedule information from said schedule storage means, and for transmitting said first schedule information to said living-body information terminal, said living-body information terminal adapted to execute said first schedule information;
detection means for detecting said execution result of said living-body information terminal following said first schedule information from data transmitted from said living-body information terminal; and
schedule updating means for generating said second schedule information in response to said execution result detected by said detection means.

56. A health-care device that communicates with a living-body information terminal adapted to detect health condition information concerning a health condition of a user, or to issue action directions to said user to take action regarding a medical treatment, comprising:
schedule storage means for storing a first schedule information for said living-body information terminal to detect a health condition information of said user or to issue an action direction to said user to take an action, and for storing a third schedule information to be executed by said living-body information terminal;
communication means for reading said first schedule information from said schedule storage means, and for transmitting said first schedule information to said living-body information terminal, said living-body information terminal adapted to execute said first schedule information;
reception means for receiving said direction of said external input; and
schedule updating means for generating said third schedule information in response to said direction received by said reception means, said third schedule information transmitted to said living-body information terminal by said communication means.

57. The health-care device according to claim 56, further comprising
detection means for detecting an execution result of said living-body information terminal following said third schedule information, from data transmitted from said living-body information terminal, and
wherein said schedule updating means generates a new schedule information in response to said execution result detected by said detection means.

58. The healthcare device according to claim 55 or claim 57,
wherein said detection means is further adapted to detect whether or not said action was executed by said user.

59. The healthcare device according to claim 58,
wherein said detection means is further adapted to detect the time of when said action was executed by said user.

60. The healthcare device according to claim 55 or claim 57,
wherein said detection means is further adapted to detect said health condition information resulting from said living-body information terminal following said first schedule information or said third schedule information.

61. The healthcare device according to claim 60,
wherein said detection means is further adapted to detect the time of when said health condition information is detected.

62. The healthcare device according to claim 55 or claim 57,
wherein said detection means is further adapted to detect an input information from an input means for inputting said input information disposed on said living-body information terminal.

63. The healthcare device according to claim 62,
wherein said detection means is further adapted to detect the time of when said input information is detected.

64. The healthcare device according to claim 55 or claim 57,
wherein said detection means is adapted to determine whether or not said action is completed by said user within a prefixed duration of time.

65. The healthcare device according to claim 55 or claim 56,
wherein said schedule updating means is adapted to instruct said living-body information terminal to follow said first schedule information when said second schedule information or said third schedule information is completed.

66. The healthcare device according to claim 55 or claim 56,
wherein said schedule updating means is further adapted to transmit a unique identification information associated with each of said first schedule information, said second schedule information and said third schedule information when said living-body information terminal is instructed to follow one of said first schedule information, said second schedule information and third schedule information.

67. The healthcare device according to claim 55 or claim 56,
wherein said first schedule information, said second schedule information and said third schedule information each comprises an action table information to detect the health condition information concerning said health condition of said user or to issue said action direction to said user to take said action regarding said medical treatment, and a judgment table information defining at least one operation for said medical treatment corresponding to said execution result, and
wherein said schedule updating means changes the schedule information according to the execution result following said action table information based on information defined in said judgment table information.

68. The health-care device according to claim 67, wherein, said second schedule information or said third schedule information are information of changing a part of said first schedule information.

69. A relay device in a health-care system in which at least one living-body information terminal is connected with said relay device, said relay device is connected with a managing device via a telecommunications network, comprising:
schedule storage means for storing a first schedule information for said living-body information terminal to detect a health condition information or to issue an action direction to take an action, and for storing a second schedule information to be executed by said at least one living-body information terminal in response to an execution result of said at least one living-body information terminal following said first schedule information;
communication means for reading said first schedule information from said schedule storage means, and for transmitting said first schedule information to said at least one living-body information terminal, said at least one living-body information terminal adapted to execute said first schedule information;
detection means for detecting said execution result of said at least one living-body information terminal following said first schedule information from data transmitted from said at least one living-body information terminal; and
schedule updating means for generating said second schedule information in response to said execution result detected by said detection means.

70. A relay device in a health-care system in which at least one living-body information terminal is connected with said relay device, said relay device is connected with a managing device via a telecommunications network, comprising:
schedule storage means for storing a first schedule information for said at least one living-body information terminal to detect a health condition information or to issue an action direction to take an action, and for storing a third schedule information to be executed by said at least one living-body information terminal;
communication means for reading said first schedule information from said schedule storage means, and for transmitting said first schedule information to said at least one living-body information terminal, said at least one living-body information terminal adapted to execute said first schedule information;
reception means for receiving said direction of said external input; and
schedule updating means for generating said third schedule information in response to said direction received by said reception means, said third schedule information transmitted to said living-body information terminal by said communication means.

71. A managing device in a health-care system in which at least one living-body information terminal is connected with a relay device and said relay device is connected with said managing device via a telecommunications network, comprising:
schedule storage means to store a first schedule information for said at least one living-body information terminal to detect a health condition information or to issue an action direction to take an action, and for storing a second schedule information to be executed by said at least one living-body information terminal in response to an execution result of said at least one living-body information terminal following said first schedule information;
communication means for reading said first schedule information from said schedule storage means, and for transmitting said first schedule information to said at least one living-body information terminal, said at least one living-body information terminal adapted to execute said first schedule information;
detection means for detecting said execution result of said at least one living-body information terminal following said first schedule information from the data transmitted from said at least one living-body information terminal through said relay device; and
schedule updating means for generating said second schedule information in response to said execution result detected by said detection means.

72. A managing device in a health-care system in which at least one living-body information terminal is connected with a relay device and said relay device is connected with said managing device via a telecommunications network, comprising:
schedule storage means for storing a first schedule information for said at least one living-body information terminal to detect a health condition information or to issue an action direction to take an action, and for storing a third schedule information to be executed by said at least one living-body information terminal;
communication means for reading said first schedule information from said schedule storage means, and for transmitting said first schedule information to said at least one living-body information terminal via the said relay device, said at least living-body information terminal adapted to execute said first schedule information;
reception means for receiving said direction of said external input; and
schedule updating means for generating said third schedule information in response to said direction received by said reception means, said third schedule information transmitted to said living-body information terminal by said communication means.

73. A living-body information terminal for detecting health condition information concerning the health condition of a user or for issuing action directions to said user to take action regarding a medical treatment, said living-body information terminal adapted to communicate with a healthcare device, comprising:
communication means for receiving a first schedule information and a second schedule information from said health-care device;
schedule execution management means for detecting a health condition information or for issuing an action direction to said user to take an action according to said first schedule information or said second schedule information received by said communication means;
detection means for detecting an execution result of said schedule execution management means detecting said health condition information or issuing said action direction; and
schedule updating means for changing the schedule information to be executed by said schedule execution management means from said first schedule information to said second schedule information in accordance with said execution result detected by said detection means.

74. A living-body information terminal for detecting health condition information concerning the health condition of a user, or for issuing action directions to said user to take action regarding a medical treatment, said living-body information terminal adapted to communicate with a healthcare device, comprising:
communication means for receiving a first schedule information and a third schedule information from said health-care device;
schedule execution management means for detecting a health condition information or for issuing an action direction to said user to take an action according to said first schedule information received by said communication means;
reception means for receiving an external direction; and
schedule updating means for changing the schedule information to be executed by said schedule execution management means from said first schedule information to said third schedule information in accordance with said external direction received by said reception means.

75. The living-body information terminal according to claim 74, wherein
said schedule execution management means is further adapted for detecting said health condition information or issuing said action direction to said user to take said action according to said third schedule information, and wherein said living-body information terminal further comprises detection means for detecting an execution result of said schedule execution management means following said third schedule information, and
wherein said schedule updating means is further adapted to change the schedule information to be executed by said schedule execution management means according to the execution result detected by said detection means.

76. The living-body information terminal according to claim 73 or claim 75, wherein said detection means is further adapted to detect whether or not said action was executed by said user.

77. The living-body information terminal according to claim 76, wherein said detection means is further adapted to detect the time of when said action was executed by said user.

78. The living-body information terminal according to claim 73 or claim 75, wherein said detection means is further adapted to detect said health condition information resulting from said living-body information terminal following said first schedule information or said third schedule information.

79. The living-body information terminal according to claim 78, wherein said detection means is further adapted to detect the time of when said health condition information is detected.

80. The living-body information terminal according to claim 73 or claim 75, wherein said detection means is further adapted to detect an input information from an input means for inputting said input information disposed on said living-body information terminal.

81. The living-body information terminal according to claim 80, wherein said detection means is further adapted to detect the time of when said input information is detected.

82. The living-body information terminal according to claim 73 or claim 75, wherein said detection means is adapted to determine whether or not said action is completed by said user within a prefixed duration of time.

83. The living-body information terminal according to claim 73 or claim 75, wherein said detection means is further adapted to detect one of or both of pulse rates and blood sugar value of said user, and the time when one of or both of said pulse rates and said blood sugar value are detected.

84. The living-body information terminal according to claim 83, wherein said schedule updating means is adapted to instruct said living-body information terminal to follow said first schedule information when said second schedule information or said third schedule information is completed.

85. The living-body information terminal according to claim 73 or claim 75, wherein said schedule updating means is further adapted to receive a unique identification information associated with each of said first schedule information, said second schedule information and said third schedule information transmitted by said healthcare device when said living-body information terminal is instructed by said healthcare device to follow one of said first schedule information, said second schedule information and third schedule information.

86. The living-body information terminal according to claim 73 or claim 75, wherein said first schedule information, said second schedule information and said third schedule information each comprises an action table information to detect the health condition information concerning said health condition of said user or to issue said action direction to said user to take said action regarding said medical treatment, and a judgment table information defining at least one operation for said medical treatment corresponding to said execution result, and wherein said schedule updating means changes the schedule information according to the execution result following said action table information based on information defined in said judgment table information.

87. The living-body information terminal according to claim 86, wherein said second schedule information or said third schedule information are information of changing a part of said first schedule information.

88. The living-body information terminal according to claim 86, further comprising judgment request means for requesting said healthcare device to judge when said execution result of said schedule execution management means following said first schedule information, said second schedule information or said third schedule information is judged abnormal using said judgment table information.

89. A living-body information terminal, comprising means for transmitting a schedule information or an execution result of said schedule information to at least one other living-body information terminal, and further comprising means for displaying said schedule information or said execution result received from said at least one other living-body information terminal.

90. A health-care system, comprising:
at least one living-body information terminal connected to a relay device wherein said relay device is connected to a managing device via a telecommunications network, and wherein when said living-body information terminal is called a lower side device, and said managing device is called an upper side device, said upper side device capable of storing information concerning schedule information stored in said at least one lower side device and schedule information stored in said at least one lower side device that has been changed by said upper side device.

91. A health-care system in which a health-care device connects with a living-body information terminal that detects health condition information concerning the health condition of a user, or issues action directions for said user to take action regarding medical treatment comprising:
a health-care device, comprising:
schedule storage means for storing a first schedule information for said living-body information terminal to detect a health condition information of said user or to issue an action direction to said user to take an action, and for storing a second schedule information to be executed by said living-body information terminal in response to an execution result of said living-body information terminal following said first schedule information;
communication means for reading said first schedule information from said schedule storage means, and for transmitting said first schedule information to said living-body information terminal, said living-body information terminal adapted to execute said first schedule information;
detection means for detecting said execution result of said living-body information terminal following said first schedule information from data transmitted from said living-body information terminal; and
schedule updating means for generating said second schedule information in response to said execution result detected by said detection means; and
a living-body information terminal, comprising:
communication means for receiving a first schedule information and a second schedule information from said health-care device;
schedule execution management means for detecting a health condition information or for issuing an action direction to said user to take an action according to said first schedule information or said second schedule information received by said communication means;
detection means for detecting an execution result of said schedule execution management means detecting said health condition information or issuing said action direction; and
schedule updating means for changing the schedule information to be executed by said schedule execution management means from said first schedule information to said second schedule information in accordance with said execution result detected by said detection means.

92. A health-care system in which a health-care device connects with a living-body information terminal that detects health condition information concerning the health information of a user, or issues action directions to let said user take action regarding medical treatment, comprising:
a health-care device, comprising:
schedule storage means for storing a first schedule information for said living-body information terminal to detect a health condition information of said user or to issue an action direction to said user to take an action, and for storing a third schedule information to be executed by said living-body information terminal;
communication means for reading said first schedule information from said schedule storage means, and for transmitting said first schedule information to said living-body information terminal, said living-body information terminal adapted to execute said first schedule information;
reception means for receiving said direction of said external input; and
schedule updating means for generating said third schedule information in response to said direction received by said reception means, said third schedule information transmitted to said living-body information terminal by said communication means; and
a living-body information terminal, comprising:
communication means for receiving a first schedule information and a third schedule information from said health-care device;
schedule execution management means for detecting a health condition information or for issuing an action direction to said user to take an action according to said first schedule information received by said communication means;
reception means for receiving an external direction; and
schedule updating means for changing the schedule information to be executed by said schedule execution management means from said first schedule information to said third schedule information in accordance with said external direction received by said reception means.

93. A schedule management method in a health-care system having a living-body information terminal to detect health condition information concerning the health condition of a user, or to issue action directions to said user to take action regarding a medical treatment, said method comprising the steps of:
detecting a health condition information of said user or issuing an action direction to said user to take an action based on a first schedule information;
detecting a detection result of said health condition information, or a result of said action direction; and
changing the schedule information to be executed according to said detection result from said first schedule information to a second schedule information.

94. A schedule management method in a health-care system having a living-body information terminal to detect health condition information concerning the health condition of a user, or to issue action directions to said user to take action regarding medical treatment, said method comprising the steps of:
detecting a health condition information of said user or issuing an action direction to said user to take an action based on a first schedule information;
receiving an external direction; and
changing the schedule information to be executed according to said external direction from said first schedule information to a third schedule information.

95. A schedule management method used in a relay device in a health-care system in which said relay device communicates with a living-body information terminal to detect health condition information concerning the health condition of a user or to issue action directions to said user to take action regarding a medical treatment, and is connected with a managing device via a telecommunications network, said method comprising the steps of:
transmitting a first schedule information to said living-body information terminal to detect a health condition information of said user or to issue an action direction to said user to take an action, and instructing said living-body information terminal to execute said first schedule information;
detecting an execution result of said living-body information terminal following said first schedule information from data transmitted from said living-body information terminal; and
changing the schedule information to be executed by said living-body information terminal from said first schedule information to a second schedule information in accordance to said execution result.

96. A schedule management method used in a relay device in a health-care system in which said relay device communicates with a living-body information terminal to detect health condition information concerning the health condition of a user or to issue action directions to said user to take action regarding a medical treatment, and is connected with a managing device via a telecommunications network, said method comprising the steps of:
transmitting a first schedule information which indicates a schedule for said living-body information terminal to detect a health condition information of said user or to issue an action direction to said user to take an action, and instructing said living-body information terminal to execute said first schedule information;
receiving an external direction; and
changing said schedule information to be executed by said living-body information terminal from said first schedule information to a third schedule information in accordance to said external direction.

97. A schedule management method used in a managing device in a health-care system in which a relay device communicates with a living-body information terminal to detect health condition information concerning the health condition of a user or to issue action directions to said user to take action regarding medical treatment, and is connected with the managing device via a network, said method comprising the steps of:
transmitting a first schedule information to said living-body information terminal to detect a health condition information of said user or to issue an action direction to said user to take an action, and instructing said living-body information terminal to execute said first schedule information;
detecting an execution result of said living-body information terminal following said first schedule information from data transmitted from said living-body information terminal via said relay device; and
changing the schedule information to be executed by said living-body information terminal via said relay device from said first schedule information to the second schedule information in accordance to said execution result.

98. A schedule management method used in a managing device in a health-care system in which a relay device communicates with a living-body information terminal to detect health condition information concerning the health condition of a user or to issue action directions to said user to take action regarding medical treatment, and is connected with the managing device via a telecommunications network, said schedule management method comprising the steps of:
transmitting a first schedule information which indicates a schedule for said living-body information terminal to detect a health condition information of said user or to issue an action direction to said user to take an action, and instructing said living-body information terminal to execute said first schedule information;
receiving an external direction; and
changing said schedule information to be executed by said living-body information terminal via said relay device from said first schedule information to the third schedule information in accordance to said external direction.

99. A schedule management method used in a living-body information terminal in a health-care system in which a relay device communicates with said living-body information terminal to detect health condition information concerning the health condition of a user or to issue action directions to said user to take action regarding a medical treatment, and is connected with a managing device via a telecommunications network, said method comprising the steps of:
detecting a health condition information of said user or issuing an action direction to said user to take an action based on a first schedule information;
detecting an execution result of said health condition information, or the result of said action direction; and
changing the schedule information to be executed by said living-body information terminal from said first schedule information to a second schedule information in accordance with said execution result.

100. A schedule management method used in a living-body information terminal in a health-care system in which a relay device communicates with said living-body information terminal to detect health condition information concerning the health condition of a user or to issue action directions to said user to take action regarding a medical treatment, and is connected with the managing device via a network, said method comprising the steps of:
detecting a health condition information of said user or issuing an action direction to said user to take an action based on a first schedule information;
receiving an external direction; and
changing the schedule information to be executed by said living-body information terminal from said first schedule information to a third schedule information in accordance with said external direction.

101. A schedule management program in a health-care system with a living-body information terminal to detect health condition information concerning the health condition of a user, or to issue action directions to said user to take action regarding a medical treatment, said schedule management program making a computer execute the steps of:
detecting a health condition information of said user or issuing an action direction to said user to take an action based on a first schedule information;
detecting a detection result of said health condition information, or a result of said action direction; and
changing the schedule information to be executed according to said detection result from said first schedule information to a second schedule information.

102. A schedule management program in a health-care system with a living-body information terminal to detect health condition information concerning the health condition of a user, or to issue action directions to said user to take action regarding a medical treatment, said schedule management program making a computer execute the steps of:
detecting a health condition information of said user or issuing an action direction to said user to take an action based on a first schedule information;
receiving an external direction; and
changing the schedule information to be executed according to said external direction from said first schedule information to a third schedule information.

103. A schedule management program used in a relay device in a health-care system in which the relay device, communicating with a living-body information terminal to detect health condition information concerning the health condition of a user or to issue action directions to said user to take action regarding a medical treatment, is connected with a managing device via a network, said schedule management program making a computer execute the steps of:
transmitting a first schedule information to said living-body information terminal to detect a health condition information of said user or to issue an action direction to said user to take an action, and instructing said living-body information terminal to execute said first schedule information;
detecting an execution result of said living-body information terminal following said first schedule information from data transmitted from said living-body information terminal; and
changing the schedule information to be executed by said living-body information terminal from said first schedule information to a second schedule information in accordance to said execution result.

104. A schedule management program used in a relay device in a health-care system in which the relay device, communicating with a living-body information terminal to detect health condition information concerning the health condition of a user or to issue action directions to said user to take action regarding a medical treatment, is connected with a managing device via a network, said schedule management program making a computer execute the steps of:
transmitting a first schedule information which indicates a schedule for said living-body information terminal to detect a health condition information of said user or to issue an action direction to said user to take an action, and instructing said living-body information terminal to execute said first schedule information;
receiving an external direction; and
changing said schedule information to be executed by said living-body information terminal from said first schedule information to a third schedule information in accordance to said external direction.

105. A schedule management program used in a managing device in a health-care system in which a relay device, communicating with a living-body information terminal to detect health condition information concerning the health condition of a user or to issue action directions to said user to take action regarding a medical treatment, is connected with the managing device via a network, said schedule management program making a computer execute the steps of:
transmitting a first schedule information to said living-body information terminal to detect a health condition information of said user or to issue an action direction to said user to take an action, and instructing said living-body information terminal to execute said first schedule information;
detecting an execution result of said living-body information terminal following said first schedule information from data transmitted from said living-body information terminal via said relay device; and
changing the schedule information to be executed by said living-body information terminal via said relay device from said first schedule information to the second schedule information in accordance to said execution result.

106. A schedule management program used in a managing device in a health-care system in which a relay device, communicating with a living-body information terminal to detect health condition information concerning the health condition of a user or to issue action directions to said user to take action regarding a medical treatment, is connected with the managing device via a network, said schedule management program making a computer execute the steps of:
transmitting a first schedule information which indicates a schedule for said living-body information terminal to detect a health condition information of said user or to issue an action direction to said user to take an action, and instructing said living-body information terminal to execute said first schedule information;
receiving an external direction; and
changing said schedule information to be executed by said living-body information terminal via said relay device from said first schedule information to the third schedule information in accordance to said external direction.

107. A schedule management program used in a living-body information terminal in a health-care system in which a relay device, communicating with the living-body information terminal to detect health condition information concerning the health condition of a user or to issue action directions to said user to take action regarding a medical treatment, is connected with a managing device via a network, said schedule management program making a computer execute the steps of:
detecting a health condition information of said user or issuing an action direction to said user to take an action based on a first schedule information;
detecting an execution result of said health condition information, or the result of said action direction; and
changing the schedule information to be executed by said living-body information terminal from said first schedule information to a second schedule information in accordance with said execution result.

108. A schedule management program used in a living-body information terminal in a health-care system in which a relay device, communicating with the living-body information terminal to detect health condition information concerning the health condition of a user or to issue action directions to said user to take action regarding a medical treatment, is connected with a managing device via a network, said schedule management program making a computer execute the steps of:
detecting a health condition information of said user or issuing an action direction to said user to take an action based on a first schedule information;
receiving an external direction; and
changing the schedule information to be executed by said living-body information terminal from said first schedule information to a third schedule information in accordance with said external direction.
